SEQUENCE LISTING (3 sequences)

SEQ ID NO: 1 - protein, 467 aa, Homo sapiens
MLLLLLLPLLWGRERVEGQKSNRKDYSLTMQSSVTVQEGMCVHVRCSFSYPVDSQTDSDPVHGYWFRAGNDISWKAPVATNNPAWAVQEETRDRFHLLGDPQTKNCTLSIRDARMSDAGRYFFRMEKGNIKWNYKYDQLSVNVTALTHRPNILIPGTLESGCFQNLTCSVPWACEQGTPPMISWMGTSVSPLHPSTTRSSVLTLIPQPQHHGTSLTCQVTLPGAGVTTNRTIQLNVSYPPQNLTVTVFQGEGTASTALGNSSSLSVLEGQSLRLVCAVDSNPPARLSWTWRSLTLYPSQPSNPLVLELQVHLGDEGEFTCRAQNSLGSQHVSLNLSLQQEYTGKMRPVSGVLLGAVGGAGATALVFLSFCVIFIVVRSCRKKSARPAADVGDIGMKDANTIRGSASQGNLTESWADDNPRHHGLAAHSSGEEREIQYAPLSFHKGEPQDLSGQEATNNEYSEIKIPK

SEQ ID NO: 2 - protein, 463 aa, Homo sapiens
MLLLLLPLLWGRERAEGQTSKLLTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSLQSKATSGVTQGVVGGAGATALVFLSFCVIFVVVRSCRKKSARPAAGVGDTGIEDANAVRGSASQGPLTEPWAEDSPPDQPPPASARSSVGEGELQYASLSFQMVKPWDSRGQEATDTEYSEIKIHR

SEQ ID NO: 3 - protein, 464 aa, Macaca fascicularis
MLLLLLLLPLLWGRERVEGQRNNQKNYPLTMQESVTVQQGLCVHVLCSFSYPWYGWISSDPVHGYWFRAGAHTDRDAPVATNNPARAVREDTRDRFHLLGDPQTKNCTLSIRDARSSDAGTYFFRVETGKTKWNYKYVPLSVHVTALTHRPNILIPGTLESGCPRNLTCSVPWACEQGTAPMISWMGTSVSPLDPSTTRSSVLTLIPQPQDHGTSLTCQVTFPGASVTTNKTIHLNVSYPPQNLTMTVFQGNDTVSIVLGNGSSVSVPEGPSLRLVCAVDSNPPARLSLSWGGLTLCPSQPSNPGVLELPRVHLRDEEEFTCRAQNLLGSQRVSLNVSLQSKATSGLTQGAVGAGATALVFLSFCVIFVVVRSCRKKSARPVAGVGDVGIEDANAVRGSASQGSLTEPWAEDSPPDQPPPASARSSVGEEELQYASLSFQTVKPRDLQGQEATNTEYSEIKIHK

(19) United States
(12) Patent Application Publication
(10) Pub. No.: US 2021/0253695 A1
(43) Pub. Date: Aug. 19, 2021

(54) USE OF SIGLEC-7 OR SIGLEC-9 ANTIBODIES FOR THE TREATMENT OF CANCER

(71) Applicant: Palleon Pharmaceuticals Inc., Waltham, MA (US)

(72) Inventors: James W. Broderick, Wellesley, MA (US); Karl D. Normington, Prides Crossing, MA (US)

(21) Appl. No.: 17/080,132

(22) Filed: Oct. 26, 2020

Related U.S. Application Data
(63) Continuation of application No. 16/069,582, filed on Jul. 12, 2018, now abandoned.
(60) Provisional application No. 62/387,985, filed on Jan. 12, 2016.

(51) Int. Cl. C07K 16/28; A61P 35/00

(52) U.S. Cl. CPC C07K 16/2803; A61K 2039/505; A61P 35/00

(57) ABSTRACT

The present application provides compositions and methods for treating a patient with cancer, and in particular epithelial tumors and carcinomas, with antibodies directed to CD33-like Siglecs.

### CROSS REFERENCE TO RELATED APPLICATIONS

This application claims the benefit of, and priority to, U.S. Provisional Patent Application Ser. No. 62/387,985 filed Jan. 12, 2016, which is hereby incorporated by reference herein in its entirety.

### FIELD OF THE INVENTION

The invention relates generally to compositions and methods for the treatment of cancer, for example, epithelial tumors and carcinomas, and more specifically the invention relates to compositions containing antibodies directed to CD33-like Siglecs and their use in the treatment of cancer, for example, epithelial tumors and carcinomas.

### BACKGROUND OF THE INVENTION

Siglecs (Sialic acid-binding immunoglobulin-type lectins) are cell surface proteins that bind sialic acid. Siglecs comprise a lectin family of surface receptors that bind to sialoglycans and are predominantly expressed on cells of the hematopoietic system in a manner dependent on cell type and differentiation. There are 14 different mammalian Siglecs, providing an array of different functions based on cell surface receptor-ligand interactions. These receptor-glycan interactions can mediate, among other things, cell adhesion and cell signaling. Although sialic acid is ubiquitously expressed, typically at the terminal position of glycoproteins and lipids, only very specific, distinct sialoglycan structures are recognized by individual Siglecs, depending on identity and linkage to subterminal carbohydrate moieties.

Siglecs are Type I transmembrane proteins where the amino terminus is located in the extracellular space and the carboxy terminus is located in the cytosol. Each Siglec contains an N-terminal V-type immunoglobulin domain (Ig domain) that acts as the binding receptor for sialic acid. Siglecs are lectins, and are categorized into the group of I-type lectins because the lectin domain is an immunoglobulin fold. All Siglecs extend from the cell surface by means of intervening C2-type Ig domains which have no binding activity. Siglecs differ in the number of these C2-type domains. As these proteins contain Ig domains, they are members of the Immunoglobulin superfamily (IgSF).

Most Siglecs, and in particular the CD33-like Siglecs, contain immunoreceptor tyrosine-based inhibitory motifs (ITIMs) in their cytosolic region. These act to down-regulate signaling pathways involving phosphorylation, such as those induced by immunoreceptor tyrosine-based activation motifs (ITAMs).

Due to their ITIM-containing cytoplasmic regions, most CD33-like Siglecs interfere with cellular signaling, thereby inhibiting immune cell activation. Once bound to their ligands, these Siglecs recruit inhibitory proteins such as SHP phosphatases via their ITIM domains. The tyrosine contained within the ITIM becomes phosphorylated upon ligand binding and acts as a docking site for SH2 domain-containing proteins like SHP phosphatases. This leads to dephosphorylation of cellular proteins, and down-regulating activating signaling pathways.

Siglecs have been attractive therapeutic targets because of their cell type-specific expression pattern, endocytic properties, high expression on certain lymphomas/leukemias, and ability to modulate receptor signaling. To date, Siglec-targeting based therapies have involved antibody- and glycan-based strategies that directly target tumor cells. Several antibody-based therapies directly targeting Siglecs on the surface of malignant cells currently are undergoing clinical evaluation and continue to be developed for the treatment of lymphoma/leukemia and autoimmune disease (Angata et al. (2015), TRENDS IN PHARMACOLOGICAL SCIENCES, 36, 10, 645-660).

A growing body of evidence supports roles for glycans, and sialoglycans in particular, at various pathophysiological steps of tumor progression. Glycans regulate tumor proliferation, invasion, haematogenous metastasis and angiogenesis (Fuster et al. (2005) NAT. REV. CANCER 5(7):526-42). The sialylation of cell surface glycoconjugates is frequently altered in cancers, resulting in the expression of sialylated tumor-associated carbohydrate antigens that are specific markers for this disease. Because sialylated glycans are involved in many biological processes, their expression by tumor cells is often associated with increased aggressiveness and metastatic potential of the tumors.

Mucins are a family of high molecular weight, heavily glycosylated and sialylated proteins (glycoconjugates) produced by epithelial tissues. A key characteristic of mucins is their ability to form gels; therefore they are a key component in most gel-like secretions, having functions from lubrication to cell signaling to forming chemical barriers. They often take an inhibitory role. Certain mucins are associated with controlling mineralization, including nacre formation in mollusks, calcification in echinoderms and bone formation in vertebrates. They bind to pathogens as part of the immune system. Overexpression of the mucin proteins, for example, MUC1, is associated with many types of cancer (Niv Y (2008) WORLD J. GASTROENTEROL. 14 (14):2139-41).

At least 19 human mucin genes have been identified by cDNA cloning—MUC1, MUC2, MUC3A, MUC3B, MUC4, MUC5AC, MUC5B, MUC6, MUC7, MUC8, MUC12, MUC13, MUC15, MUC16, MUC17, MUC19, and MUC20 (Perez-Vilar, J; Hill, R L (2004) “Mucin Family of Glycoproteins,” ENCYCLOPEDIA OF BIOLOGICAL CHEMISTRY (Lennarz & Lane, Eds.) (Oxford: Academic Press/Elsevier) 2: 758-764).

Increased mucin production occurs in many adenocarcinomas, including cancers of the pancreas, lung, breast, ovary, colon and other tissues. Mucins are also overexpressed in lung diseases such as asthma, bronchitis, chronic obstructive pulmonary disease (COPD) or cystic fibrosis. Two membrane mucins, MUC1 and MUC4 have been extensively studied in relation to their pathological implication in the disease process. Mucins are under investigation as possible diagnostic markers for malignancies and other disease processes in which they are most commonly over- or mis-expressed.

For example, MUC16 is a cell surface mucin expressed at high levels by epithelial ovarian tumors. Following proteolytic cleavage, cell surface MUC16 (csMUC16) is shed extracellularly and is detected in the serum of cancer patients as the tumor marker CA125. It is believed that csMUC16 acts as an adhesion molecule and facilitates peritoneal metastasis of ovarian tumors.

The innate immune system, also known as the nonspecific immune system, is an important subsystem of the overall immune system that comprises the cells and mechanisms that defend the host from infection by other organisms. The cells of the innate system recognize and respond to pathogens in a generic way, but, unlike the adaptive immune system (which is found only in vertebrates), it does not confer long-lasting or protective immunity to the host. Cells of the innate immune system include natural killer cells (NK cells), mast cells, eosinophils, basophils and phagocytic cells including macrophages, neutrophils, and dendritic cells.

A fundamental tenet of the immune system is the requirement for immune cells to respond to transformed or infected cells while remaining tolerant of normal cells. NK cells are a component of the innate immune system that does not directly attack invading microbes. Rather, NK cells destroy compromised host cells, such as tumor cells or virus-infected cells, recognizing such cells by a condition known as “missing self,” which is a term used to describe cells with abnormally low levels of a cell-surface marker called major histocompatibility complex class I (MHC I), a situation that can arise in viral infections of host cells. The NK cells were named “natural killer” because of the initial belief that they do not require activation in order to kill cells that are “missing self” For many years, it was unclear how NK cell recognized tumor cells and infected cells. It is now understood that the MHC makeup on the surface of those cells is altered and the NK cells become activated through recognition of “missing self” Normal body cells are not recognized and attacked by NK cells because they express intact self MHC antigens. Those MHC antigens are recognized by killer cell immunoglobulin receptors (KIR) that, in essence, put the brakes on NK cells (Kumar et al. (2005) NAT. REV. IMMUNOL. 5(5):363-74).

In vivo, a tumor is significantly more complex than a simple group of clonogenic cells. The three-dimensional mass that is appreciated on imaging studies contains, in addition to tumor cells, extracellular matrix components, supportive stromal cells (e.g. neovasculature, fibroblasts, and macrophages), and a number of inflammatory cells. In terms of mounting an anti-tumor immune response, there is further complexity because the priming and effector phases of the immune response are separated by time and space. While priming occurs in lymph nodes, the effector functions must operate within the tumor mass. Potential barriers to anti-tumor responses encountered during the priming phase include a paucity of “danger” signals from innate immune cells, poor recruitment of dendritic cells (DCs) for cross-presentation, and inadequate expression of costimulatory ligands on tumor cells or antigen presenting cells (APCs). Potential barriers to efficacy during the effector phase involve inadequate recruitment of activated immune cells to abnormal vascular endothelial cells and/or chemokines, the presence of dominant immune inhibitory mechanisms capable of abrogating T cell effector function (e.g. the inhibitory receptors PD-1 and CTLA-4), extrinsic suppressive cells (TREGs, myeloid-derived suppressor cells), metabolic inhibitors (IDO, arginase), and inhibitory cytokines (IL-10, TGF-β).

The genetic profile of the tumor microenvironment and its potential correlation with anti-tumor immune responses has become an area of increased study in recent years. For instance, the use of antibody based therapies directed to PD-1 and/or PD-L1 have been shown to have a dramatic effect on T-cell recognition and increased clearance of tumor cells.

Despite the advances made in immunooncology, there is still a need for additional therapeutic treatments.

### SUMMARY OF THE INVENTION

The present invention provides compositions and methods for treating a subject with cancer, and in particular epithelial tumors and carcinomas, with antibodies directed to CD33-like Siglecs. In addition, the present invention provides methods and compositions using anti-Siglec antibodies with preferred activities. In particular, the invention provides anti- CD33-like

Siglec antibodies which bind CD33-like Siglecs expressed on the surface of cells involved in innate and adaptive immunity, that are capable of interfering with Siglec recognition of their sialic acid based ligands are useful for treating or preventing cancer. Use of the antibodies to block the Siglec binding to their natural ligands interferes with the suppression of the innate immune system that is a hallmark of cancer.

In one aspect, the invention provides a method of treating or reducing the risk of cancer in a subject in need thereof, the method comprising administering to the subject a therapeutically effective amount of an antibody to a CD33-like Siglec, thereby to treat the cancer. In certain embodiments, the antibody interferes with the CD33-like Siglec binding to a sialic acid ligand. In certain embodiments, the antibody interferes with the suppression of cells of the innate immune response directed to the cancer. In certain embodiments, the CD33-like Siglec is expressed on the surface of a cell of the innate immune system.

In certain embodiments, the Siglec is a CD33 related Siglec (CD33rSiglec), such as, for example, Siglec-3, Siglec-5, Siglec-6, Siglec-7, Siglec-8, Siglec-9, Siglec-10, Siglec-11, or other polypeptides encoded by nucleic acid sequences identified as Siglec sequences. In certain embodiments, the Siglec is Siglec-7. In certain embodiments, the Siglec is Siglec-9.

In another aspect, the invention provides a method of treating cancer in a subject in need thereof, the method comprising administering to the subject a therapeutically effective amount of an anti-Siglec-7 or anti-Siglec-9 antibody thereby to treat the cancer, wherein the antibody interferes with the binding of Siglec-7 or Siglec-9 to the its respective sialic acid ligand. In certain embodiments, the antibody interferes with the suppression of cells of the innate immune response directed to the cancer. In certain embodiments, wherein the Siglec-7 or Siglec-9 is expressed on the surface of a cell of the innate immune system.

In certain embodiments, the natural ligands of the Siglec include a sialoglycoprotein expressed on the surface of a cancer cell. In other embodiments, the natural ligands of the Siglec include a sialoglycoprotein secreted by a cancer cell. Most Siglec based sialic acid ligands are bonded via the 2, 3, 6 and occasionally 8 hydroxyl groups (number dependent on the carbon to which they are attached), in an α anomeric configuration. In some embodiments, sialic acid linkage specificities include α2,3, α2,6, and α2,8.

In certain embodiments, the antibody that binds Siglec-7 binds preferentially to an epitope selected from the group consisting of amino acid residues 19-32 of SEQ ID NO: 1, amino acid residues 47-76 of SEQ ID NO: 1, amino acid residues 47-62 of SEQ ID NO: 1 and amino acid residues 63-76 of SEQ ID NO: 1.

In certain embodiments, the antibody that binds Siglec-9 binds preferentially to an epitope selected from the group consisting of amino acid residues 18-27 of SEQ ID NO: 2, amino acid residues 42-72 of SEQ ID NO: 2, amino acid residues 42-58 of SEQ ID NO: 2, amino acid residues 59-72 of SEQ ID NO: 2, amino acid residues 20-33 of SEQ ID NO: 3, amino acid residues 48-77 of SEQ ID NO: 3, amino acid residues 48-63 of SEQ ID NO: 3, and amino acid residues 64-77 of SEQ ID NO:3.

In certain embodiments, cells of the innate immune system that express Siglecs, and in particular CD33-like Siglecs, include, for example, myeloid progenitors, monocytes, neutrophils, natural killer (NK) cells, dendritic cells, macrophages, eosinophils. In other embodiments, cells of the adaptive immune cells that express Siglecs include, for example, B cells and T cells.

In certain embodiments, the antibody inhibits the binding of the natural Siglec ligand to the Siglec, resulting in interfering with the normal suppression or inhibition of innate and adaptive immunity. In certain embodiments, antibody binding blocks the ability of the Siglec to bind a sialoglycoprotein. In other embodiments, binding of preferred antibodies blocks the ability of the Siglec to suppress the innate immune response. In other embodiments, antibody binding blocks the ability of the Siglec to suppress the adaptive immune response. In other embodiments, antibody binding blocks the ability of the Siglec to recruit inhibitory proteins such as SHP phosphatases via their ITIM domains.

In certain embodiments, the antibody inhibits ligand dependent crosslinking, ligation and/or clustering of CD33 related Siglecs. In certain embodiments, the antibody inhibits ligand dependent endocytosis of CD33 related Siglecs.

In certain embodiments, the antibody reduces or inhibits ligand dependent calcium mobilization within a cell. In certain embodiments, the antibody blocks activation of a nuclear factor of activated T-cells (NFAT) response within the cell.

In certain embodiments, the modulation can be by inhibition of the interaction between Siglecs on the immune cells and a sialoglycan on epithelial cancers. The modulation can be mediated by inhibiting the ability of Siglecs on the immune cells to bind to sialic based ligands on the surface of the epithelial cancers. Alternatively, a modulation can be mediated by inhibiting the ability of the Siglecs on the immune cells to bind sialic acid based ligands secreted by epithelial cancers. Examples of sialic based ligands produced by epithelial cancers include mucins.

In certain embodiments the epithelial cancer upregulates the expression sialylated glycans (hypersialylation). In certain embodiments the epithelial cancer includes, but is not limited to, endometrial cancer, colon, ovarian cancer, cervical cancer, vulvar cancer, uterine cancer or fallopian tube cancer, breast cancer, prostate cancer, lung cancer, pancreatic cancer, urinary cancer, bladder cancer, head and neck cancer, oral cancer and liver cancer. Epithelial cancers also include carcinomas, for example, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, baso squamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, and carcinoma villosum.

It is understood that the invention provides any combination of two, three, or more of the foregoing embodiments in each aspect of the invention.

### DETAILED DESCRIPTION

During malignant transformation, glycosylation is heavily altered compared to healthy, non-cancerous tissue due to differential expression of glycosyltransferases, glycosidases and monosaccharide transporters within the cancer microenvironment. One key change of malignant tissue glycosylation is the alteration of sialic acid processing that leads to a general upregulation of sialylated glycans (hypersialylation) on cell surfaces. These changes may result from altered sialyltransferase, and sialidase expression. Functionally, cancer-associated hypersialylation appears to directly impact tumor cell interaction with the microenvironment, in particular the modulation of sialic acid-binding lectins on immune cells.

### I. Siglecs

### Siglec-3

CD33 or Siglec-3 is a transmembrane receptor expressed on cells of myeloid lineage. It is usually considered myeloid-specific, but it can also be found on some lymphoid cells. The extracellular portion of this receptor contains two immunoglobulin domains (one IgV and one IgC2 domain), placing Siglec-3 within the immunoglobulin superfamily. The intracellular portion of Siglec-3 contains immunoreceptor tyrosine-based inhibitory motifs (ITIMs) that are implicated in inhibition of cellular activity.

Tyrosine phosphorylation of the two intra-cellular tyrosine-based motifs of CD33 by antibody cross-linking of the receptor or by treatment with the protein tyrosine phosphatase inhibitor pervanadate has been shown to result in the recruitment of several SH2 domain-containing proteins such as SHP-1 and SHP-2, Syk, CrkL, and PLC-γ1. Based on mutagenesis studies, the membrane-proximal ITIM motif appears to be dominant in CD33 interactions with the inhibitory tyrosine phosphatases SHP-1 and SHP-2. CD33 tyrosine phosphorylation appears to depend on Src family kinases, and the Src kinase Lck has been shown to be effective at phosphorylating the proximal, but not the distal, tyrosine residue of human CD33. Protein tyrosine phosphatases associated with CD33 appear to catalyze CD33 dephosphorylation, suggesting a potential inhibitory feedback control of CD33 signaling. After coligation, CD33 has been shown to inhibit Fc-gamma receptor RI (FcγRI)-induced calcium mobilization, in the manner of a classical inhibitory receptor.

Antibody crosslinking of CD33 results in inhibition of growth of normal CD34+ myeloid progenitor cells and leukemic cells isolated from the blood of patients suffering from chronic myeloid leukemia (CML), as well as inhibition of proliferation and induction of apoptosis in leukemic cells from acute myeloid leukemia (AML) patients and some AML cell lines. Subsequent proliferation studies revealed that cells from 56% of patients were responsive to the inhibitory effects of anti-CD33 antibody crosslinking, while 44% were unresponsive. The latter correlated with reduced Syk and/or zeta chain-associated protein kinase 70 (ZAP-70) expression levels or function, pointing to significant biochemical differences between responder and nonresponder AML populations.

CD33 also has been shown to have inhibitory functions on mature myeloid cells. For instance, incubation with CD33 antibody dramatically reduced the survival not only of human CD34+ myeloid progenitor cells, but also of dendritic cells derived from monocytes in vitro, even though mature dendritic cells were resistant to these effects. CD33 appears to inhibit human monocyte production of pro-inflammatory cytokines, such as IL-1β, TNF-α, and IL-8 via phosphatidylinositol 3-kinase (PI3K) and p38 mitogen-activated protein kinase (MAPK). Furthermore, suppressor of cytokine signaling 3 (SOCS3) has recently been shown to associate with the phosphorylated ITIM motif of CD33 resulting in its proteosomal degradation and inhibition of the effect of CD33 engagement on cytokine-induced proliferation. Finally, it has been demonstrated that when ITIMs, such as those found in CD33, are phosphorylated by tyrosine kinases, they can bind to the ubiquitin ligase Cbl and become ubiquitylated, leading to CD33 internalization.

CD33, like other Siglecs, has been shown to prefer α2-6-linked sialic acid-containing glycans. Pretreatment of transfected COS-cells with neuraminidase is typically required for CD33-dependent binding to red blood cells, which are heavily sialylated on their surface, with subsequent rosette formation, suggesting that the binding activity of CD33 can be masked by endogenous carbohydrates on the surface of the same cell. Mutations in the proximal tyrosine motif of CD33 or inhibition of CD33 phosphorylation with pharmacological agents have resulted in an increase of sialic acid-dependent rosette formation, suggesting that CD33 signaling through selective recruitment of SHP-1/SHP-2 may modulate its ligand binding activity.

Siglec-3 was the target of gemtuzumab ozogamicin, a monoclonal antibody-based treatment for acute myeloid leukemia, which was subsequently voluntarily withdrawn from the market in 2010. However, anti-CD33 monoclonal antibodies have been and continue to be used for the diagnosis of various types of AML.

### Siglec-5

Sialic acid-binding Ig-like lectin 5 (Siglec-5) is a protein that in humans is encoded by the SIGLEC5 gene. SIGLEC5 has also been designated CD170 (cluster of differentiation 170). Like other family members, Siglec-5 also appears to act as an inhibitory receptor. For example, primary human T cells, which do not express Siglec-5, show reduced T cell receptor responsiveness after transfection with Siglec-5. In a transfected rat basophilic leukemia (RBL) model system, Siglec-5 could efficiently recruit SHP-1 and SHP-2 after tyrosine phosphorylation and inhibit calcium flux and serotonin release after co-ligation with the ITAM-containing high-affinity IgE receptor (FcεRI). Mutagenesis studies suggested that inhibition of serotonin release could still occur efficiently after a double tyrosine-to-alanine substitution in the membrane proximal ITIM and the membrane distal ITIM-like motif. A potential mechanism for tyrosine phosphorylation-independent inhibitory signaling was supported by results of in vitro phosphorylation assays, which is associated with activation of SHP-1 by the Siglec-5 cytoplasmic tail in the absence of tyrosine phosphorylation. Increased Siglec-5 expression observed on alveolar macrophages and macrophages within reactive lymph nodes activated by bacterial or viral pathogens, suggest that macrophages, upon activation during normal clearance activity in the lung and upon immune responses in lymph nodes, respectively, upregulate Siglec-5 surface expression.

### Siglec-6

Sialic acid-binding Ig-like lectin 6 (Siglec-6) is a protein that in humans is encoded by the SIGLEC6 gene and binds to alpha-2,6-linked sialic acid.

Siglec-6 was originally identified both as a leptin-binding protein (obesity binding protein-1 or OB-BP1) and as a placental protein. Based on mRNA screening, the highest levels were found in placenta, and antibody-based analyses confirmed expression on cyto- and syncytiotrophoblasts along with one of its preferred ligands, α2-6-linked sialosides, on placenta and uterine epithelium. Lower levels of mRNA expression were also detected in spleen, leukocytes, and small intestine. The leukocyte and splenic sources of Siglec-6 were traced to B cells, but subsequent studies found high levels of Siglec-6 on CD34+-derived human mast cells and the HMC-1 mast cell line, but not basophils or lung mast cells.

### Siglec-7

Sialic acid-binding Ig-like lectin 7 (Siglec-7) is a protein that in humans is encoded by the SIGLEC7 gene (Nicoll Get al. (1999) J. BIOL. CHEM. 274 (48): 34089-95). SIGLEC7 has also been known as cluster of differentiation 328 (CD328).

Siglec-7 is found on NK cells. Siglec-7 leads to cellular inactivation once bound to its sialic acid-containing cognate ligand and is found in high levels on NK cell surfaces. Siglec-7 is understood to mediate cell-cell contacts by binding to sialylated glycans on target cells leading to inhibition of NK cell-dependent killing of the target cell. Mammalian cells typically contain high levels of sialic acid and so when NK cells bind so called “self-cells,” they are not activated and do not kill host cells. Siglec-7 has been shown to preferentially recognize α2-8-linked sialylated glycans.

Various studies have suggested that Siglec-7 mediates inhibitory activity. In a rat basophilic leukemia (RBL) cell model, co-crosslinking of Siglec-7 and FcεRI inhibited serotonin release. Furthermore, site-directed mutagenesis experiments suggested that the membrane-proximal ITIM motif was essential for both the inhibitory function and the recruitment of the inhibitory phosphatases SHP-1 and SHP-2. The capacity of Siglec-7 to recruit SHP-1 after tyrosine phosphorylation has previously been observed in polyclonal NK cell populations isolated from human peripheral blood leukocytes. Siglec-7, in a manner consistent with other classical inhibitory receptors of the immune system, has the capacity to antagonize the effector function of an activating receptor by recruitment of inhibitory phosphatases to counteract tyrosine phosphorylation of an ITIM motif. Preferred antibodies to Siglec-7 likely will reduce or block the ability of Siglec-7 to antagonize the effector function of such activating receptors, thus maintaining immunorecognition of tumor cells.

### Siglec-8

Sialic acid-binding Ig-like lectin 8 (Siglec-8) is a protein that in humans is encoded by the SIGLEC8 gene. This gene is located on chromosome 19q13.4, about 330 kb downstream of the SIGLEC9 gene.

Siglec-8 is expressed by human eosinophils, mast cells, and, to a lesser extent, basophils. Siglec-8 is believed to be a molecule uniquely expressed by immune effector cells involved in asthma and allergy. In both eosinophils and mast cells, Siglec-8 is expressed late in development.

Two splice variants of Siglec-8 exist. The initially characterized form of Siglec-8 contains 431 amino acid residues in total, 47 of which comprise a short cytoplasmic tail when compared to most CD33-associated Siglecs. Subsequently, a longer form of Siglec-8, initially termed Siglec-8L, that contains 499 amino acid residues was identified. This longer form of Siglec-8 shares the same extracellular region but includes a longer cytoplasmic tail with two tyrosine-based motifs (an immunoreceptor tyrosine-based inhibitory motif (ITIM) and an immunoreceptor tyrosine-based switch motif (ITSM)). Both forms of Siglec-8 are found on eosinophils and contain a V-set domain with lectin activity and two C2-type Ig repeat domains in the extracellular region. Given that the longer version is believed to be the normal version, the term Siglec-8 is typically used to refer to the 499 amino acid version, while the 431 amino acid version is typically referred to as the “short form” of Siglec-8.

Potential glycan ligands for Siglec-8 have been screened by glycan array. The glycan NeuAcα2-3(6-O-sulfo)Galβ1-4[Fucα1-3]GlcNAc, also known as 6′-sulfo-sialyl Lewis X, binds with high affinity to both Siglec-8 and to a mouse Siglec, Siglec-F, which appears to have acquired a similar but not identical function and pattern of expression to human Siglec-8 through convergent evolution. Rescreening on a more expanded glycan array reconfirmed this finding, but also identified a second closely related ligand in which the fucose is absent (NeuAcα2-3(6-O-sulfo)Galβ1-4GlcNAc, or 6′-sulfated sialyl N-acetyl-D-lactosamine. These interactions are quite specific; no binding could be detected between these Siglecs and unsulfated sialyl Lewis X or sialyl Lewis X sulfated at carbon 6 of GlcNAc (6-sulfo-sialyl Lewis X) rather than carbon 6 of galactose as in 6′-sulfo-sialyl Lewis X. Similarly, no other

Siglecs bind effectively to these Siglec-8 ligands, as demonstrated by selective binding to eosinophils in human blood of a polymer decorated with 6′-sulfo-sialyl Lewis X. The natural ligand or ligands for Siglec-8 have not yet been positively identified, but ongoing studies have determined that there are sialidase-sensitive glycoprotein ligands for Siglec-F in mouse airways that require the activity of the α2,3 sialyltransferase 3 (ST3Gal-III) enzyme for their generation.

### Siglec-9

Sialic acid-binding Ig-like lectin 9 (Siglec-9) is a protein that in humans is encoded by the SIGLEC9 gene (Foussias Get al. (2000) GENOMICS 67 (2): 171-8). Siglec-9 is a transmembrane protein that is expressed in monocytes, granuloctyes and macrophages, and has an extracellular domain, a transmembrane domain and a cytoplasm domain.

Siglec-9 is highly expressed on neutrophils and monocytes, and at lower levels on subpopulations of T and B lymphocytes and NK cells. Siglec-9 protein expression is absent from eosinophils, and little or no Siglec-9 mRNA is produced in normal hematopoietic progenitor cells or human mast cells derived by culture of CD34+ peripheral blood precursors. Immature neutrophils gain Siglec-9 surface expression late in differentiation, appearing after the myelocyte stage but before CD16 is expressed. Siglec-9 is highly expressed on AML cells at levels similar to CD33, particularly on a subset with myelomonoblastic features. Like CD33, Siglec-9 mediates rapid endocytosis of bound specific antibody. Antibodies to Siglec-9 of the present invention can block the endocytosis of Siglec-9, which can be mediated by engagement with multivalent sialylated ligands or by antibodies capable of crosslinking or ligating Siglec-9.

Similar to other members of the CD33 subfamily, Siglec-9 contains two cytoplasmic tyrosine motifs: a membrane-proximal ITIM and a distal ITIM-like domain. Cell activation or exposure to cytokines within minutes leads to rapid tyrosine phosphorylation of Siglec-9 by tyrosine kinases. Upon tyrosine phosphorylation, Siglec-9 recruits the inhibitory phosphatases SHP-1 and SHP-2. Functional evidence that Siglec-9 acts as an inhibitory receptor comes from RBL cell transfectants, where co-crosslinking with FcεRI inhibited serotonin release. Site-directed mutagenesis experiments suggest that the classical membrane-proximal ITIM motif is essential for this inhibitory function. Siglec-9 was also shown to be capable of negative regulation of T cell receptor signaling using Jurkat cells transfected with these receptors. The finding that cell activation by TCR engagement increased tyrosine phosphorylation and recruitment of SHP-1 is suggestive for a negative feedback role.

Similar to Siglec-8, ligation of Siglec-9 by antibodies induced apoptosis in human neutrophils. Antibodies to Siglec-9 of the present invention can block the induction of Siglec-9, which can be mediated by engagement with multivalent sialylated ligands or by antibodies capable of crosslinking or ligating Siglec-9.

Siglec-9-mediated cell death may be enhanced by cytokines such as granulocyte/macrophage colony-stimulating factor (GM-CSF), interferon-α (IFN-α), and IFN-γ, and “primed” neutrophils from patients with sepsis or rheumatoid arthritis may be more susceptible to Siglec-9-mediated death than normal cells. Incubation with GM-CSF can result in rapid tyrosine phosphorylation of Siglec-9 and increase the potency and efficacy of Siglec-9-dependent death upon antibody crosslinking. The increased Siglec-9-mediated death may be caspase independent, but yet dependent on reactive oxygen species, which may be referred to as a non-apoptotic, autophagy-like morphology. The observation that cytokine priming might recruit alternative death pathways suggests a complex interplay between cytokine receptor and Siglec signaling pathways. Siglec-9 shows affinity for α2-3-, α2-6-, and α2-8-linked sialosides but a preference for α2-8-linked sialosides.

Alteration of the surface glycosylation pattern on malignant cells potentially affects tumor immunity by directly influencing interactions with lectins (glycan-binding proteins) on the surface of immunomodulatory cells. The sialic acid-binding Ig-like lectins Siglec-7 and -9 are MHC class I-independent inhibitory receptors on human NK cells that recognize sialic acid containing carbohydrates. Siglec-9 defines a subset of cytotoxic NK cells with a mature phenotype and enhanced chemotactic potential. Interestingly, this Siglec-9+ NK cell population was reduced in the peripheral blood of cancer patients. Broad analysis of primary tumor samples has revealed that ligands of Siglec-7 and -9 are expressed on human cancer cells of different histological types. Expression of Siglec-7 and -9 ligands was associated with susceptibility of NK cell-sensitive tumor cells and of presumably NK cell-resistant tumor cells to NK cell-mediated cytotoxicity.

### Siglec-10

Sialic acid-binding Ig-like lectin 10 (Siglec-10) is a protein that in humans is encoded by the SIGLEC10 gene. The mouse orthologue is Siglec G. Like most but not all other Siglecs, Siglec-10 bears an immunoreceptor tyrosine-based inhibitory motif (ITIM) within its cytoplasmic domain. Siglec-10 is a ligand for CD52, the target of the therapeutic monoclonal antibody alemtuzumab. It is also reported to bind to vascular adhesion protein 1 (VAP-1) and to the co-stimulatory molecule CD24 also known as heat-stable antigen (HSA).

Sialoconjugates with α2-3 and α2-6 linkage appear to be recognized by Siglec-10, with a preference for sialoconjugates with an α2-6 linkage. In kinase assays Siglec-10 was phosphorylated in decreasing order by Lck, Jak3, and Emt, but not ZAP-70. SHP-1 and SHP-2 have been shown to associate with Siglec-10, whereas no interaction with the SH2-protein SLAM-associated protein (SAP) has been observed.

### Siglec-11

The SIGLEC-11 protein (686 amino acids) is expressed in various organs (Angata et al. (2002) J. BIOL. CHEM 277(27): 24466-74). Intense staining is observed in chronically inflamed tissues. The protein does not seem to be expressed in peripheral blood leukocytes. Expression has been observed in Kupffer cells, intestinal lamina propria macrophages, microglial cells, perifollicular cells of the spleen. SIGLEC-11 binds specifically to alpha-2-8-linked sialic acids.

### Siglec Ligands

Magesh et al. (2011) CURR. MED. CHEM. 18(23):3537-50 discusses certain aspects of the structure and function of Siglec receptors, and describes the identification of sialic acid based high-affinity ligands of certain Siglecs.

As described in O'Reilly et al. (2010) METHODS ENZYMOL. 478: 343-363, synthetic sialoside ligands of Siglecs have been developed to probe their function and glycan-binding specificity, and to detect Siglecs on different cell types. Specificity can be considered from the perspective of the Siglec and of the carbohydrate ligand, which may also have one or more cognate binding partners. CD22 is highly specific for sialosides with the alpha-2,6 linkage, but other more promiscuous Siglecs can bind this sialoside as well, precluding specific targeting of this sequence to CD22. Siglec-7 appears to exhibit a preference for glycans with the NeuAcα2,8-NeuAcα2,3-Galβ1,4-GlcNAc sequence, but also bind NeuAcα2,3-Galβ1,4-GlcNAc and NeuAcα2,6-Galβ1,4-GlcNAc. Siglec-8 expressed on eosinophils appears to bind preferentially to 6′-sulfo-sialyl LewisX. As an example of specificity from the perspective of the ligand, a polyacrylamide polymer of 6′-sulfo-sialyl LewisX binds selectively to only eosinophils among leukocytes in a sample of whole blood.

### II. Anti-Siglec Antibodies

The term “antibody” includes polyclonal antibodies, monoclonal antibodies (including full length antibodies which have an immunoglobulin Fc region), antibody compositions with polyepitopic specificity, multispecific antibodies (e.g., bispecific antibodies, diabodies, and single-chain molecules, as well as antibody fragments (e.g., Fab, F(ab′)₂, and Fv). The terms “immunoglobulin” or “Ig” are used interchangeably with “antibody” herein.

The basic 4-chain antibody unit is a heterotetrameric glycoprotein composed of two identical light (L) chains and two identical heavy (H) chains. An IgM antibody contains 5 of the basic heterotetramer units along with an additional polypeptide called a J chain, and contains 10 antigen binding sites, while IgA antibodies comprise from 2-5 of the basic 4-chain units which can polymerize to form polyvalent assemblages in combination with the J chain. In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H chain has at the N-terminus, a variable domain (VH) followed by three constant domains (CH) for each of the alpha and gamma chains and four CH domains for mu and epsilon isotypes. Each L chain has at the N-terminus, a variable domain (VL) followed by a constant domain at its other end. The VL is aligned with the VH and the CL is aligned with the first constant domain of the heavy chain (CH1). Particular amino acid residues are believed to form an interface between the light chain and heavy chain variable domains. The pairing of a VH and VL together forms a single antigen-binding site. For the structure and properties of the different classes of antibodies, see e.g., Basic and Clinical Immunology, 8th Edition, Daniel P. Sties, Abba I. Ten and Tristram G. Parsolw (Eds.), Appleton & Lange, Norwalk, Conn., 1994, page 71 and Chapter 6.

The L chain from any vertebrate species can be assigned to one of two clearly distinct types, called kappa and lambda, based on the amino acid sequences of their constant domains. Depending on the amino acid sequence of the constant domain of their heavy chains (CH), immunoglobulins can be assigned to different classes or isotypes. There are five classes of immunoglobulins: IgA, IgD, IgE, IgG and IgM, having heavy chains designated alpha, beta, epsilon, gamma and mu, respectively. The gamma and alpha classes are further divided into subclasses on the basis of relatively minor differences in the CH sequence and function, e.g., humans express the following subclasses: IgG1, IgG2, IgG3, IgG4, IgA1 and IgA2. IgG1 antibodies can exist in multiple polymorphic variants termed allotypes any of which are suitable for use in the invention. Common allotypic variants in human populations are those designated by the letters a, f, n, and z.

An “isolated” antibody is one that has been identified, separated and/or recovered from a component of its natural environment and/or production environment (e.g., naturally or recombinantly). In some embodiments, the isolated polypeptide is free of association with other components from its natural environment and/or production environment. For example, an isolated protein may be substantially free of cellular material or other proteins from the cell or tissue source from which it is derived. The term “isolated” also refers to preparations where the isolated protein is sufficiently pure to be administered as a pharmaceutical composition, or at least 70-80% (w/w) pure, more preferably, at least 80-90% (w/w) pure, even more preferably, 90-95% pure; and, most preferably, at least 95%, 96%, 97%, 98%, 99%, or 100% (w/w) pure. In some embodiments, the polypeptide is purified: (1) to greater than 95% by weight of antibody as determined by, for example, the Lowry method, and in some embodiments, to greater than 99% by weight; (1) to a degree sufficient to obtain at least 15 residues of N-terminal or internal amino acid sequence by use of a spinning cup sequenator, or (3) to homogeneity by SDS-PAGE under non-reducing or reducing conditions using Coomassie blue or silver stain. Isolated antibody includes the antibody in situ within recombinant cells since at least one component of the antibody's natural environment will not be present. Ordinarily, however, an isolated polypeptide or antibody is prepared by at least one purification step.

The term “naked antibody” refers to an antibody that is not conjugated to a cytotoxic moiety or radiolabel.

The terms “full-length antibody,” “intact antibody” or “whole antibody” are used interchangeably to refer to an antibody in its substantially intact form, as opposed to an antibody fragment. Specifically whole antibodies include those with heavy and light chains including an Fc region. The constant domains may be native sequence constant domains (e.g., human native sequence constant domains) or amino acid sequence variants thereof. In some cases, the intact antibody may have one or more effector functions.

An “antibody fragment” comprises a portion of an intact antibody, the antigen binding and/or the variable region of the intact antibody. Examples of antibody fragments include Fab, Fab′, F(ab′)₂ and Fv fragments; diabodies; linear antibodies (see U.S. Pat. No. 5,641,870, Zapata et al. (1995) PROTEIN ENG. 8(10): 1057-1062); single-chain antibody molecules and multispecific antibodies formed from antibody fragments.

The Fc fragment comprises the carboxy-terminal portions of both H chains held together by disulfides. The effector functions of antibodies are determined by sequences in the Fc region, the region which is also recognized by Fc receptors (FcR) found on certain types of cells.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is (are) identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al. (1984) PROC. NATL. ACAD. SCI. USA, 81:6851-6855). As used herein, “humanized antibody” is used as a subset of “chimeric antibodies.”

“Humanized” forms of non-human (e.g., murine) antibodies are chimeric antibodies that contain minimal sequence derived from non-human immunoglobulin. In one embodiment, a humanized antibody is a human immunoglobulin (recipient antibody) in which residues from a human variable region of the recipient are replaced by residues from a non-human species (donor antibody) such as mouse, rat, rabbit or non-human primate having the desired specificity, affinity, and/or capacity. In some instances, framework residues (FR) of the human immunoglobulin are replaced by corresponding non-human residues. These modifications may be made to further refine antibody performance, such as binding affinity. For example, a humanized antibody may comprise substantially all of at least one, and typically two, variable domains, in which all or substantially all of the hypervariable loops corresponding to those of a non-human immunoglobulin sequence, and all or substantially all of the FR regions are those of a human immunoglobulin sequence, although the FR regions may include one or more individual FR residue substitutions that improve antibody performance, such as binding affinity, isomerization, immunogenicity, etc. The humanized antibody optionally also comprises at least a portion of an immunoglobulin constant region (Fc), typically that of a human immunoglobulin. For further details, see, e.g., Jones et al. (1986) NATURE 321:522-525; Riechmann et al. (1988) NATURE 332:323-329; and Presta (1992) CURR. OP. STRUCT. BIOL. 2:593-596. In certain embodiments, humanized antibodies are directed against a single antigenic site. In certain embodiments, humanized antibodies are directed against multiple antigenic sites. Alternative humanization protocols are described in U.S. Pat. No. 7,981,843.

Generally, antibodies comprise six hypervariable regions (HVRs) or complementary determining regions (CDRs), for example, three in the VH (referred to as CDR_{H1}, CDR_{H2}, and CDR_{H3}) and three in the VL (referred to as CDR_{L1}, CDR_{L2}, and CDR_{L3}). Naturally occurring camelid antibodies consisting of a heavy chain only are functional and stable in the absence of light chain. See, e.g., Hamers-Casterman et al. (1993) NATURE 363:446-448 and Sheriff et al. (1996) NATURE STRUCT. BIOL. 3:733-736.

Antibody “effector functions” refer to those biological activities attributable to the Fc region (a native sequence Fc region or amino acid sequence variant Fc region) of an antibody, and vary with the antibody isotype. Examples of antibody effector functions include: C1q binding and complement dependent cytotoxicity; Fc receptor binding; antibody-dependent cell-mediated cytotoxicity (ADCC); phagocytosis; down regulation of cell surface receptors (e.g., B cell receptors); and B cell activation.

“Antibody-dependent cell-mediated cytotoxicity” or “ADCC” refers to a form of cytotoxicity in which secreted Ig bound onto Fc receptors (FcRs) present on certain cytotoxic cells (e.g., NK cells, neutrophils and macrophages) enable these cytotoxic effector cells to bind specifically to an antigen-bearing target cell and subsequently kill the target cell with cytotoxins. The antibodies can activate the cytotoxic cells and are required for killing of the target cell by this mechanism. The primary cells for mediating ADCC, NK cells, express FcgRIII only, whereas monocytes express FcgRT, FcgRII and FcgRIII. In certain embodiments, an anti-Siglec antibody described herein enhances ADCC. To assess ADCC activity of a molecule of interest, an in vitro ADCC assay, such as that described in U.S. Pat. Nos. 5,500,362 or 5,821,337 may be performed. Useful effector cells for such assays include peripheral blood mononuclear cells (PBMC) and NK cells. Alternatively, or additionally, ADCC activity of the molecule of interest may be assessed in vivo, e.g., in an animal model such as that disclosed in Clynes et al. (1998) PNAS USA 95:652-656. Other Fc variants that alter ADCC activity and other antibody properties include those disclosed by Ghetie et al. (1997) NAT. BIOTECH. 15:637-40; Duncan et al. (1988) NATURE 332:563-564; Lund et al. (1991) J. IMMUNOL. 147:2657-2662; Lund et al. (1992) MOL. IMMUNOL. 29:53-59; Alegre et al. (1994) TRANSPLANTATION 57:1537-1543; Hutchins et al. (1995) PROC NATL. ACAD SCI USA 92:11980-11984; Jefferis et al. (1995) IMMUNOL. LETT. 44:111-117; Lund et al. (1995) FASEB J9:115-119; Jefferis et al. (1996) IMMUNOL. LETT. 54:101-104; Lund et al. (1996) J. IMMUNOL. 157:4963-4969; Armour et al. (1999) EUR. J. IMMUNOL. 29:2613-2624; Idusogie et al. (2000) J. IMMUNOL. 164:4178-4184; Reddy et al. (2000) J. IMMUNOL. 164:1925-1933; Xu et al. (2000) CELL. IMMUNOL. 200:16-26; Idusogie et al. (2001) J. IMMUNOL. 166:2571-2575; Shields et al. (2001) J. BIOL. CHEM. 276:6591-6604; Jefferis et al. (2002) IMMUNOL. LETT. 82:57-65; Presta et al. (2002) BIOCHEM. SOC. TRANS. 30:487-490; Lazar et al. (2006) PROC. NATL. ACAD. SCI. USA 103:4005-4010; U.S. Pat. Nos. 5,624,821; 5,885,573; 5,677,425; 6,165,745; 6,277,375; 5,869,046; 6,121,022; 5,624,821; 5,648,260; 6,194,551; 6,737,056; 6,821,505; 6,277,375; 7,335,742; and 7,317,091.

Suitable native-sequence Fc regions for use in the antibodies of the invention include human IgG1, IgG2, IgG3 and IgG4. A single amino acid substitution (S228P according to Kabat numbering; designated IgG4Pro) may be introduced to abolish the heterogeneity observed in recombinant IgG4 antibody. See Angal, S. et al. (1993) MOL. IMMUNOL. 30:105-108.

In certain embodiments, the antibody has a human IgG4 isotype or another isotype that elicits little or no antibody-dependent cell-mediated cytotoxicity (ADCC) and/or complement mediated cytotoxicity (CDC). In certain embodiments, the antibody has a human IgG4 isotype.

The terms “modulate,” “immunomodulatory,” and their cognates refer to a reduction or an increase in the activity of Siglec associated with downregulation of T cell responses due to its interaction with an anti-Siglec antibody, wherein the reduction or increase is relative to the activity of Siglec in the absence of the same antibody. A reduction or an increase in activity is preferably at least about 10%, 20%, 30%, 40%, 50%, 60%, 70%, 80%, 90%, or more, relative to the activity in the absence of the antibody in question. When Siglec activity is reduced, the terms “modulatory” and “modulate” are interchangeable with the terms “inhibitory” and “inhibit.” When Siglec activity is increased, the terms “modulatory” and “modulate” are interchangeable with the terms “activating” and “activate.”

The terms “crosslinking”, “ligation” and/or “clustering” refer to the response of CD33 related Siglecs on the cell surface in the presence of multivalent sialylated ligands. In some embodiments, clustering, ligation or crosslinking refers to more than 10 CD33 related Siglecs of the same kind interacting with each other. In other embodiments, clustering, ligation or crosslinking refers to more than 9, or 8, or 7, or 6, or 5 or 4 CD33 related Siglecs of the same kind interacting with each other. An antibody of the present invention blocks or interferes or reduces crosslinking, ligation and/or clustering of CD33 related Siglecs on the cell surface in the presence of multivalent sialylated ligands.

The term “inhibition or reduction of endocytosis” refers to a reduction in the number of CD33 related Siglecs on the cell surface in the presence of multivalent sialylated ligands. An antibody of the present invention may inhibit or reduce endocytosis of CD33 related Siglecs on the cell surface in the presence of multivalent sialylated ligands. A reduction or inhibition of endocytosis is preferably at least about 10%, 20%, 30%, 40%, 50%, 60%, 70%, 80%, 90%, or more of that induced by multivalent sialylated ligand engagement in the absence of the antibody in question.

The term “block”, “reduce”, “antagonize” with respect to the ability of a ligand to bind to a Siglec by an antibody of the present invention refers to a lowering of the ligand induced response upon binding to a Siglec. This lowering can be through direct competition of a ligand by an antibody of the present invention. Alternatively, lowering can be through alternative means such as steric hindrance of ligand induced signaling, inhibition of multimerization of Siglecs on the cell membrane. The ability of an antibody to block, reduce or antagonize is preferably at least about 10%, 20%, 30%, 40%, 50%, 60%, 70%, 80%, 90%, or more of that induced by multivalent sialylated ligand engagement in the absence of the antibody in question.

The term “bind preferentially”, as used in connection with an antibody refers to an antibody that reacts or associates more frequently, more rapidly, with greater duration and/or with stronger affinity with a particular target molecule (e.g., a protein, carbohydrate, glycoprotein, or glycolipid) than it does with alternative target molecules. For example, an antibody that specifically or preferentially binds to a Siglec epitope is an antibody that binds this epitope with stronger affinity, avidity, more readily, and/or with greater duration than it binds to other Siglec epitopes or non-Siglec epitopes. The antibody may have affinity for the target molecule stronger than 100 nM, 50 nM, 20 nM, 15 nM, 10 nM, 9 nM, 8 nM, 7 nM, 6 nM, 5 nM, 4 nM, 3 nM, 2 nM, or 1 nM, as determined by surface plasmon resonance. It is understood that a molecule (e.g., an antibody) that binds preferentially to a first target may or may not preferentially bind to a second target. As such, “preferential binding” does not necessarily require (although it can include) exclusive binding.

Antibodies described herein are prepared using techniques available in the art for generating antibodies, exemplary methods of which are described in more detail in the following sections.

### Antibody Fragments

The present invention encompasses antibody fragments. Antibody fragments may be generated by traditional means, such as enzymatic digestion, or by recombinant techniques. In certain circumstances there are advantages of using antibody fragments, rather than whole antibodies. For a review of certain antibody fragments, see Hudson et al. (2003) NAT. MED. 9:129-134.

Various techniques have been developed for the production of antibody fragments. Traditionally, these fragments were derived via proteolytic digestion of intact antibodies (see, e.g., Morimoto et al. (1992) JOURNAL OF BIOCHEMICAL AND BIOPHYSICAL METHODS 24:107-117; and Brennan et al. (1985) SCIENCE 229:81). However, these fragments can now be produced directly by recombinant host cells. Fab, Fv and ScFv antibody fragments can all be expressed in and secreted from E. coli, thus allowing the facile production of large amounts of these fragments. Antibody fragments can be isolated from the antibody phage libraries discussed above. Alternatively, Fab′-SH fragments can be directly recovered from E. coli and chemically coupled to form F(ab′)₂ fragments (Carter et al. (1992) BIO/TECHNOLOGY 10:163-167). According to another approach, F(ab′)₂ fragments can be isolated directly from recombinant host cell culture. Fab and F(ab′)₂ fragment with increased in vivo half-life comprising salvage receptor binding epitope residues are described in U.S. Pat. No. 5,869,046. Other techniques for the production of antibody fragments will be apparent to the skilled practitioner. In certain embodiments, an antibody is a single chain Fv fragment (scFv). See U.S. Pat. Nos. 5,571,894; and 5,587,458.

### Humanized Antibodies

In certain embodiments, an antibody molecule is a humanized antibody. A humanized antibody refers to an immunoglobulin comprising a human framework region and one or more CDRs from a non-human, e.g., mouse or rat, immunoglobulin. The immunoglobulin providing the CDRs is often referred to as the “donor” and the human immunoglobulin providing the framework often called the “acceptor,” though in embodiments, no source or no process limitation is implied. Typically a humanized antibody comprises a humanized light chain and a humanized heavy chain immunoglobulin.

### Human Antibodies

Human anti-Siglec antibodies of the invention can be constructed by combining Fv clone variable domain sequence(s) selected from human-derived phage display libraries with known human constant domain sequences(s). Alternatively, human monoclonal anti-Siglec antibodies of the invention can be made by the hybridoma method. Human myeloma and mouse-human heteromyeloma cell lines for the production of human monoclonal antibodies have been described, for example, by Kozbor (1984) J. IMMUNOL. 133:3001; Brodeur et al. (1987) MONOCLONAL ANTIBODY PRODUCTION TECHNIQUES AND APPLICATIONS, pp. 51-63 (Marcel Dekker, Inc., New York); and Boerner et al. (1991) J. IMMUNOL. 147:86.

It is possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al. (1993) PROC. NATL. ACAD. SCI. USA 90:2551; Jakobovits et al. (1993) NATURE 362: 255; Bruggermann et al. (1993) YEAR IN IMMUNOL. 7:33.

Gene shuffling can also be used to derive human antibodies from non-human (e.g., rodent) antibodies, where the human antibody has similar affinities and specificities to the starting non-human antibody. According to this method, which is also called “epitope imprinting,” either the heavy or light chain variable region of a non-human antibody fragment obtained by phage display techniques as described herein is replaced with a repertoire of human V domain genes, creating a population of non-human chain/human chain scFv or Fab chimeras. Selection with antigen results in isolation of a non-human chain/human chain chimeric scFv or Fab wherein the human chain restores the antigen binding site destroyed upon removal of the corresponding non-human chain in the primary phage display clone, i.e., the epitope governs the choice of the human chain partner. When the process is repeated in order to replace the remaining non-human chain, a human antibody is obtained (see PCT Publication No. WO93/06213). Unlike traditional humanization of non-human antibodies by CDR grafting, this technique provides completely human antibodies, which have no FR or CDR residues of non-human origin.

### Bispecific Antibodies

Bispecific antibodies are monoclonal antibodies that have binding specificities for at least two different antigens. In certain embodiments, bispecific antibodies are human or humanized antibodies. In certain embodiments, one of the binding specificities is for Siglec and the other is for any other antigen. In certain embodiments, bispecific antibodies may bind to two different epitopes of Siglec, for instance to two different CD33 related Siglecs. The two different Siglecs can be selected from Siglec-3, Siglec-5, Siglec-6, Siglec-7, Siglec-8, Siglec-9, Siglec-10, or Siglec-11. The two different Siglecs can be Siglec-9 and Siglec-7. Bispecific antibodies may also be used to localize cytotoxic agents to cells which express Siglec. Bispecific antibodies can be prepared as full length antibodies or antibody fragments (e.g., F(ab′)₂ bispecific antibodies).

Methods for making bispecific antibodies are known in the art. See Milstein and Cuello (1983) NATURE 305:537, PCT Publication No. WO93/08829, and Traunecker et al. (1991) EMBO J., 10:3655. For further details of generating bispecific antibodies see, for example, Suresh et al. (1986) METHODS ENZYMOL. 121:210. Bispecific antibodies include cross-linked or “heteroconjugate” antibodies. For example, one of the antibodies in the heteroconjugate can be coupled to avidin, the other to biotin. Heteroconjugate antibodies may be made using any convenient cross-linking method. Suitable cross-linking agents are well known in the art, and are disclosed in U.S. Pat. No. 4,676,980, along with a number of cross-linking techniques.

### Single-Domain Antibodies

In some embodiments, an antibody of the invention is a single-domain antibody. A single-domain antibody is a single polypeptide chain comprising all or a portion of the heavy chain variable domain or all or a portion of the light chain variable domain of an antibody. In certain embodiments, a single-domain antibody is a human single-domain antibody (Domantis, Inc., Waltham, Mass.; see, e.g., U.S. Pat. No. 6,248,516). In one embodiment, a single-domain antibody consists of all or a portion of the heavy chain variable domain of an antibody.

### Antibody Variants

In some embodiments, amino acid sequence modification(s) of the antibodies described herein are contemplated. For example, it may be desirable to improve the binding affinity and/or other biological properties of the antibody. Amino acid sequence variants of the antibody may be prepared by introducing appropriate changes into the nucleotide sequence encoding the antibody, or by peptide synthesis. Such modifications include, for example, deletions from, and/or insertions into and/or substitutions of, residues within the amino acid sequences of the antibody. Any combination of deletion, insertion, and substitution can be made to arrive at the final construct, provided that the final construct possesses the desired characteristics. The amino acid alterations may be introduced in the subject antibody amino acid sequence at the time that sequence is made.

A useful method for identification of certain residues or regions of the antibody that are preferred locations for mutagenesis is called “alanine scanning mutagenesis” as described by Cunningham and Wells (1989) SCIENCE 244:1081-1085. Here, a residue or group of target residues are identified (e.g., charged residues such as arg, asp, his, lys, and glu) and replaced by a neutral or negatively charged amino acid (e.g., alanine or polyalanine) to affect the interaction of the amino acids with antigen. Those amino acid locations demonstrating functional sensitivity to the substitutions then are refined by introducing further or other variants at, or for, the sites of substitution. Thus, while the site for introducing an amino acid sequence variation is predetermined, the nature of the mutation per se need not be predetermined. For example, to analyze the performance of a mutation at a given site, ala scanning or random mutagenesis is conducted at the target codon or region and the expressed immunoglobulins are screened for the desired activity.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme or a polypeptide which increases the serum half-life of the antibody.

### Antibody Properties

In certain embodiments, an anti-Siglec antibody of the invention inhibits binding of a CD33-like Siglec, e.g., Sigelc-7 or Siglec-9, to its cognate ligand. For example, in certain embodiments, an anti-Siglec antibody of the invention inhibits binding of a CD33-like Siglec, e.g., Sigelc-7 or Siglec-9, to erythrocytes, e.g., by 10%, 20%, 30%, 40%, 50%, 60%, 70%, 80%, 90% or 100% relative to the binding in the absence of the antibody in question. Erythrocyte binding assays can be performed by incubating Chinese hamster ovary (CHO) cells stably expressing various CD33-like Siglecs with erythrocytes isolated from human blood in the presence or absence of antibody. CHO cells that express the Siglec of interest can optionally be pretreated with neuraminidase to remove background from sialylated glycoproteins on the surface of the CHO cell. After incubation, the bound RBCs are lysed with water to release hemoglobin, and hemoglobin is detected via its pseudoperoxidase activity. Alternatively, binding of human Siglec Fc chimeras to human red blood cells can be performed as described by Kelm, S. et al. (1994) CURRENT BIOLOGY 4:965. Exemplary erythrocyte binding assays are described in Example 2.

In certain embodiments, an anti-Siglec antibody of the invention inhibits binding of a CD33-like Siglec, e.g., Siglec-7 or Siglec-9, to polyacrylamide (PAA) glycoconjugates, e.g., by 10%, 20%, 30%, 40%, 50%, 60%, 70%, 80%, 90% or 100% relative to the binding in the absence of the antibody in question. PAA binding assays can be performed by incubating CHO cells stably expressing various CD33-like Siglecs with PAA glycoconjugates (2,3-PAA, NeuAca2,3Galb1,4Glc coupled to PAA; 2,6-PAA, NeuAca2,6Galb1,4Glc coupled to PAA) as described previously (Nicoll, G. et al. (1999) J. BIOL. CHEM. 274:34089-34095). After incubation in the presence or absence of antibody, bound PAA conjugates are detected by incubation with fluorescein-streptavidin. Exemplary PAA binding assays are described in Example 2.

In certain embodiments, an anti-Siglec antibody of the invention inhibits binding of a CD33-like Siglec, e.g., Sigelc-7 or Siglec-9, to MUC1-ST, e.g., by 10%, 20%, 30%, 40%, 50%, 60%, 70%, 80%, 90% or 100% relative to the binding in the absence of the antibody in question. MUC1-ST can be prepared as previously described in Backstrom et al. (2003) BIOCHEM. J. 376:677-686. Exemplary MUC1-ST binding assays are described in Example 2.

In certain embodiments, an anti-Siglec antibody of the invention inhibits binding of a CD33-like Siglec, e.g., Sigelc-7 or Siglec-9, to a CD33-like Siglec ligand, e.g., CA15.3, CA125, MUC16, human erythrocytes, glycophorin A, osteopontin, hyaluronan, LGALS3BP, and/or cancer cell lines that express Siglec ligands.

In certain embodiments, an anti-Siglec antibody of the invention inhibits ligand-mediated activation of a CD33-like Siglec. For example, in certain embodiments, an anti-CD33-like Siglec antibody of the invention, e.g., an anti-Siglec-7 or anti-Siglec-9 antibody, inhibits ligand-mediated Siglec phosphorylation in a cell e.g., by 10%, 20%, 30%, 40%, 50%, 60%, 70%, 80%, 90% or 100% relative to the phosphorylation in the absence of the antibody in question. In certain embodiments, an anti-CD33-like Siglec antibody of the invention, e.g., an anti-Siglec-7 or anti-Siglec-9 antibody, inhibits Siglec-mediated SHP-1/2 phosphorylation in a cell e.g., by 10%, 20%, 30%, 40%, 50%, 60%, 70%, 80%, 90% or 100% relative to the phosphorylation in the absence of the antibody in question. In certain embodiments, an anti-CD33-like Siglec antibody of the invention, e.g., an anti-Siglec-7 or anti-Siglec-9 antibody, inhibits Siglec-mediated Erk1/2 phosphorylation in a cell e.g., by 10%, 20%, 30%, 40%, 50%, 60%, 70%, 80%, 90% or 100% relative to the phosphorylation in the absence of the antibody in question. In certain embodiments, an anti-CD33-like Siglec antibody of the invention, e.g., an anti-Siglec-7 or anti-Siglec-9 antibody, inhibits Siglec-mediated MEK1/2 phosphorylation in a cell e.g., by 10%, 20%, 30%, 40%, 50%, 60%, 70%, 80%, 90% or 100% relative to the phosphorylation in the absence of the antibody in question. In certain embodiments, an anti-CD33-like Siglec antibody of the invention, e.g., an anti-Siglec-7 or anti-Siglec-9 antibody, inhibits Siglec-mediated calcium flux in a cell in a cell e.g., by 10%, 20%, 30%, 40%, 50%, 60%, 70%, 80%, 90% or 100% relative to the calcium flux in the absence of the antibody in question. Ligand mediated activation of a CD33-like Siglec, including, e.g., Siglec phosphorylation, SHP-1/2 phosphorylation, calcium flux, Erk1/2 phosphorylation, MEK1/2 phosphorylation, and/or calcium flux can be assayed as follows. Cells engineered to express Siglec proteins, immune cell lines that endogenously express Siglec proteins, or primary immune cells purified from human whole blood are incubated with Siglec ligand in the presence or absence of antibody. Phosphorylated proteins can be detected using phospho-specific antibody based methods, and calcium flux can be detected using intracellular calcium reporters (e.g., Fluo-4; Life Technologies). Exemplary Siglec phosphorylation, SHP-1/2 phosphorylation, Erk1/2 phosphorylation, MEK1/2 phosphorylation, and/or calcium flux assays are described in Example 2.

In certain embodiments, an anti-CD33-like Siglec antibody of the invention, e.g., an anti-Siglec-7 or anti-Siglec-9 antibody, inhibits MUC1-ST binding to an isolated human primary monocytes or monocyte-derived macrophages e.g., by 10%, 20%, 30%, 40%, 50%, 60%, 70%, 80%, 90% or 100% relative to the binding in the absence of the antibody in question. In certain embodiments, an anti-CD33-like Siglec antibody of the invention, e.g., an anti-Siglec-7 or anti-Siglec-9 antibody, inhibits MUC1-ST mediated secretion of interleukin-6 (IL-6), macrophage-stimulating cytokine M-CSF, and/or plasminogen-activator inhibitor PAI-1 in isolated human primary monocytes or monocyte-derived macrophages e.g., by 10%, 20%, 30%, 40%, 50%, 60%, 70%, 80%, 90% or 100% relative to the secretion in the absence of the antibody in question. In certain embodiments, an anti-CD33-like Siglec antibody of the invention, e.g., an anti-Siglec-7 or anti-Siglec-9 antibody, inhibits MUC1-ST mediated induction of CD206, CD163, and/or PD-L1 in isolated human primary monocytes or monocyte-derived macrophages e.g., by 10%, 20%, 30%, 40%, 50%, 60%, 70%, 80%, 90% or 100% relative to the induction in the absence of the antibody in question. Anti-Siglec antibody effects on isolated human primary monocytes or monocyte-derived macrophages can be assayed as follows. To differentiate monocytes into macrophages, CD14+ cells are incubated with recombinant human M-CSF, recombinant human GM-CSF, or MUC1-ST. MUC1-ST binding can be assayed by flow cytometry or fluorescent microscopy following incubation of cells in the presence or absence of antibody. Interleukin-6 (IL-6), macrophage-stimulating cytokine M-CSF, and plasminogen-activator inhibitor PAI-1 can be assayed be ELISA following incubation of cells in the presence or absence of antibody. Exemplary binding, cytokine secretion, and differentiation assays using monocyte and monocyte-derived macrophages are described in Example 2.

In certain embodiments, an anti-CD33-like Siglec antibody of the invention, e.g., an anti-Siglec-7 or anti-Siglec-9 antibody, increases neutrophil killing of tumor cells, e.g., by 10%, 20%, 30%, 40%, 50%, 60%, 70%, 80%, 90% or 100% relative to the killing in the absence of the antibody in question. Neutrophil killing of target tumor cells can be assayed as follows. Human neutrophils are isolated from PBMCs and incubated with fluorescent labelled cancer cell lines (e.g., MC38GFP cells or CFSE pre-labeled LS180). Fluorescent cells are then quantified at different time points. Exemplary neutrophil-mediated tumor cell apoptosis assays are described in Example 2.

In certain embodiments, an anti-CD33-like Siglec antibody of the invention, e.g., an anti-Siglec-7 or anti-Siglec-9 antibody, inhibits tumor cell mediated recruitment of SHP-1 to a Siglec ITIM domain in a neutrophil, e.g., by 10%, 20%, 30%, 40%, 50%, 60%, 70%, 80%, 90% or 100% relative to the recruitment in the absence of the antibody in question. SHP-1 recruitment can be assayed by culturing isolated human neutrophils with LS180 or A549 tumor cells followed by immunoprecipitation of Siglecs and detection of SHP-1 by Western blot. Exemplary SHP-1 recruitment assays using neutrophils are described in Example 2.

In certain embodiments, an anti-CD33-like Siglec antibody of the invention, e.g., an anti-Siglec-7 or anti-Siglec-9 antibody, increases ADCC activity of PBMCs or NK cells toward breast cancer cells, e.g., by 10%, 20%, 30%, 40%, 50%, 60%, 70%, 80%, 90% or 100% relative to the ADCC activity in the absence of the antibody in question. Antibody-dependent cellular cytotoxicity (ADCC) can be assayed by measuring lactate dehydrogenase (LDH) release from breast cancer cells following incubation with PBMCs or NK cells in the presence or absence of antibody. LDH release can be measured using a LDH cytotoxicity assay kit (Thermo Fisher Scientific, 88954) according to the manufacturer's protocol. Exemplary ADCC assays are described in Example 2.

In certain embodiments, an anti-CD33-like Siglec antibody of the invention, e.g., an anti-Siglec-7 or anti-Siglec-9 antibody, inhibits Siglec mediated NFAT activation in T-cells, e.g., by 10%, 20%, 30%, 40%, 50%, 60%, 70%, 80%, 90% or 100% relative to the NFAT activation in the absence of the antibody in question. The ability of preferred antibodies of the present invention to modulate human T-cell activity can be tested in activated human T-cell lines engineered to express Siglec proteins. For example, such assays can be performed using Jurkat cells stably expressing a specific Siglec receptor and an NFAT reporter gene as described by Ikehara et al. (2004) J. BIOL. CHEM. 279 (41):43117. For example, Jurkat-derived cell lines are incubated with antibodies, Siglec ligand and/or cancer cells, and cell culture supernatants of Jurkat cells are collected and assayed for luciferase activity with a commercially available kit (Invivogen) and IL-2 by ELISA (R&D Systems, Inc.). Exemplary NFAT reporter assays are described in Example 2.

In certain embodiments, an anti-CD33-like Siglec antibody of the invention, e.g., an anti-Siglec-7 or anti-Siglec-9 antibody, inhibits Siglec mediated CD69, CD25 and/or IFN-γ expression in T-cells, e.g., by 10%, 20%, 30%, 40%, 50%, 60%, 70%, 80%, 90% or 100% relative to the expression in the absence of the antibody in question. CD8+T cells can be isolated from human PBMCs, labeled with eFluor 670 (eBioscience) and co-incubated with Siglec antibodies in the presence of anti-CD3/CD28 beads. The proliferation of CD8+ T cells, cell-surface expression of CD69 and CD25 and production of IFN-γ are measured by flow cytometry as described by Beatson et al. (2016) NATURE IMMUNOLOGY 17(11):1273. Exemplary proliferation, expression, and cytokine secretion assays using isolated T-cells are described in Example 2.

In certain embodiments, an anti-CD33-like Siglec antibody of the invention, e.g., an anti-Siglec-7 or anti-Siglec-9 antibody, inhibits ligand mediated crosslinking, ligation, clustering or endocytosis of Siglecs, e.g., by 10%, 20%, 30%, 40%, 50%, 60%, 70%, 80%, 90% or 100% relative to the crosslinking, ligation, clustering or endocytosis in the absence of the antibody in question. To measure cellular internalization and receptor endocytosis, cells engineered to express Siglec proteins, immune cell lines that endogenously express Siglec proteins, or primary immune cells purified from human whole blood can be used. Cells optionally treated with neuraminidase are incubated with Siglec antibodies in the presence and absence of Siglec ligand. Cells are washed, fixed, permeabilized stained with a non-ligand competing antibody that recognizes the Siglec protein (e.g., clone E10-286, available from BD Biosciences specific to Siglec-9 or clone Z176, available from Biolegend specific to Siglec-7) and visualized by fluorescence microscopy or flow cytometry. Exemplary internalization and endocytosis assays are described in Example 2.

In certain embodiments, an anti-Siglec-7 antibody of the invention inhibits NK-mediated cytotoxicity, e.g., by 10%, 20%, 30%, 40%, 50%, 60%, 70%, 80%, 90% or 100% relative to the cytotoxicity in the absence of the antibody in question. The inhibitory effects of Siglec-7 on NK-mediated cytotoxicity can be observed in cell lines and primary human cells (Falco et al. (1999) J. EXP. MED. 190:793-802; Nicoll et al. (1999) JBC 274:34089-34095). Siglec-7 binds to the ganglioside GD3, which displays α2-8-linked disialic acids. Exemplary Siglec-7 activity assays are described in Example 1.

In certain embodiments, an anti-CD33-like Siglec antibody of the invention, e.g., an anti-Siglec-7 or anti-Siglec-9 antibody, inhibits tumor growth in a humanized mouse model, e.g., by 10%, 20%, 30%, 40%, 50%, 60%, 70%, 80%, 90% or 100% relative to the tumor growth in the absence of the antibody in question.

In certain embodiments, an anti-CD33-like Siglec antibody of the invention, e.g., an anti-Siglec-7 or anti-Siglec-9 antibody, reduces tumor volume in a humanized mouse model, e.g., by 10%, 20%, 30%, 40%, 50%, 60%, 70%, 80%, 90% or 100% relative to the tumor volume in the absence of the antibody in question. Exemplary humanized mouse models include humanized CD34+ mouse models and humanized PBMC mouse models. Exemplary humanized mouse models are described in Example 4.

In certain embodiments, an anti-CD33-like Siglec antibody of the invention binds the the sialic acid binding V-set immunoglobulin domain of the CD33-like Siglec. For example, in certain embodiments an anti-Siglec-7 antibody binds an epitope within the sialic acid binding V-set immunoglobulin domain of Siglec-7, corresponding to amino acid residues 26-144 of SEQ ID NO: 1. In certain embodiments, an anti-Siglec-7 antibody binds preferentially to an epitope comprising amino acid residues 19-32 of SEQ ID NO: 1, amino acid residues 47-76 of SEQ ID NO: 1, amino acid residues 47-62 of SEQ ID NO: 1 and/or amino acid residues 63-76 of SEQ ID NO: 1. In certain embodiments an anti-Siglec-9 antibody binds preferentially to an epitope within the sialic acid binding V-set immunoglobulin domain of Siglec-9, corresponding to amino acid residues 23-144 of SEQ ID NO: 2. In certain embodiments, an anti-Siglec-9 antibody binds preferentially to an epitope comprising amino acid residues 18-27 of SEQ ID NO: 2, amino acid residues 42-72 of SEQ ID NO: 2, amino acid residues 42-58 of SEQ ID NO: 2 and/or amino acid residues 59-72 of SEQ ID NO: 2.

In certain embodiments, an anti-CD33-like Siglec antibody of the invention is cross-reactive between a human and a cynomologus monkey (cyno) CD33-like Siglec, i.e., the antibody binds to both a human Siglec and a cyno homolog of the human Siglec. The antibody may bind to the human Siglec with stronger affinity than the cyno Siglec, or may bind to the cyno Siglec with stronger affinity than the human Siglec. For example, in certain embodiments, the affinity of the antibody for the human Siglec is about 1, 2, 3, 4, 5, 10, 20, or 50 times stronger than the affinity of the antibody for the cyno Siglec, as determined by surface plasmon resonance. In certain embodiments, an anti-Siglec-7 antibody binds an epitope within the sialic acid binding V-set immunoglobulin domain of human Siglec-7, corresponding to amino acid residues 26-144 of SEQ ID NO: 1, and/or binds to an epitope within the sialic acid binding V-set immunoglobulin domain of cyno Siglec-7. In certain embodiments, an anti-Siglec-7 antibody binds preferentially to an epitope on human Siglec-7 comprising amino acid residues 19-32 of SEQ ID NO: 1, amino acid residues 47-76 of SEQ ID NO: 1, amino acid residues 47-62 of SEQ ID NO: 1 and/or amino acid residues 63-76 of SEQ ID NO: 1, and/or binds preferentially to a corresponding epitope on cyno Siglec-7. In certain embodiments, an anti-Siglec-9 antibody binds an epitope within the sialic acid binding V-set immunoglobulin domain of human Siglec-9, e.g., corresponding to amino acid residues 23-144 of SEQ ID NO: 2, and/or binds an epitope within the sialic acid binding V-set immunoglobulin domain of cyno Siglec-9, corresponding to amino acid residues 27-145 of SEQ ID NO: 3. In certain embodiments, an anti-Siglec-9 antibody binds preferentially to an epitope on human Siglec-9 comprising amino acid residues 18-27 of SEQ ID NO: 2, amino acid residues 42-72 of SEQ ID NO: 2, amino acid residues 42-58 of SEQ ID NO: 2, and/or amino acid residues 59-72 of SEQ ID NO: 2, and/or binds preferentially to an epitope on cyno Siglec-9 comprising amino acid residues 20-33 of SEQ ID NO: 3, amino acid residues 48-77 of SEQ ID NO: 3, amino acid residues 48-63 of SEQ ID NO: 3, and/or amino acid residues 64-77 of SEQ ID NO:3.

Exemplary anti-Siglec-7 antibodies include human Siglec-7/CD328 antibody (AF1138, goat IgG polyclonal) available from R&D Systems, clone #194212 (MAB1138, mouse monoclonal IgG2b) available from R&D Systems, clone #194211 (MAB11381, mouse monoclonal IgG1) available from R&D Systems, clone Z176 (A22330, mouse monoclonal IgG2b) available from Beckman Coulter, 6-434 (339202, mouse IgG1 monoclonal) available from Biolegend, goat anti-human Siglec-7 polyclonal antibody (product # PAS-47079) available from ThermoFisher Scientific, REA214 (recombinant human IgG1 monoclonal) available from Miltenyl Biotec, S7.7 (MCA5782GA, mouse IgG1 monoclonal antibody) available from BioRad, 10B2201 (MBS604764, mouse IgG1 monoclonal) available from MyBioSource, 8D8 (MBS690562, mouse IgG2 monoclonal) available from MyBioSource, 10B2202 (MBS608694, mouse IgG1 monoclonal) available from MyBioSource, and 5-386 (MBS214370, mouse IgG1 monoclonal) available from MyBioSource.

Exemplary anti-Siglec-9 antibodies are described in U.S. Pat. Nos. 8,394,382 and 9,265,826. Furthermore, exemplary anti-Siglec-9 antibodies include MAB1139 (clone #191240, mouse IgG2a monoclonal) available from R&D Systems, Inc., AF1139 (goat IgG polyclonal), available from R&D Systems, Inc., D18 (SC-34936, goat IgG polyclonal), available from Santa Cruz Biotechnology, Inc., Y-12 SC34938 (SC3-4938, goat IgG polyclonal), available from Santa Cruz Biotechnology, Inc., AB197981 (rabbit IgG polyclonal), available from Abcam, AB96545 (rabbit IgG polyclonal), available from Abcam, AB89484 (clone # MM0552-6K12 mouse IgG2 monoclonal), available from Abcam, AB130493 (rabbit IgG polyclonal), available from Abcam, AB117859 (clone # 3G8 mouse IgG1 monoclonal), available from Abcam, and monoclonal E10-286 (mouse, anti-human, isotype mouse IgG1) available from Becton Dickinson.

### III. Antibody Production

### Vector Construction

Polynucleotide sequences encoding polypeptide components of the antibody of the invention can be obtained using standard recombinant techniques. Desired polynucleotide sequences may be isolated and sequenced from antibody producing cells such as hybridoma cells. Alternatively, polynucleotides can be synthesized using nucleotide synthesizer or PCR techniques. Once obtained, sequences encoding the polypeptides are inserted into a recombinant vector capable of replicating and expressing heterologous polynucleotides in prokaryotic hosts. Many vectors that are available and known in the art can be used for the purpose of the present invention. Selection of an appropriate vector will depend mainly on the size of the nucleic acids to be inserted into the vector and the particular host cell to be transformed with the vector. Each vector contains various components, depending on its function (amplification or expression of heterologous polynucleotide, or both) and its compatibility with the particular host cell in which it resides. The vector components generally include, but are not limited to: an origin of replication, a selection marker gene, a promoter, a ribosome binding site (RBS), a signal sequence, the heterologous nucleic acid insert and a transcription termination sequence.

In general, plasmid vectors containing replicon and control sequences which are derived from species compatible with the host cell are used in connection with these hosts. The vector ordinarily carries a replication site, as well as marking sequences which are capable of providing phenotypic selection in transformed cells. For example, E. coli is typically transformed using pBR322, a plasmid derived from an E. coli species. pBR322 contains genes-encoding ampicillin (Amp) and tetracycline (Tet) resistance and thus provides easy means for identifying transformed cells. pBR322, its derivatives, or other microbial plasmids or bacteriophage may also contain, or be modified to contain, promoters which can be used by the microbial organism for expression of endogenous proteins. Examples of pBR322 derivatives used for expression of particular antibodies are described in detail in U.S. Pat. No. 5,648,237.

In addition, phage vectors containing replicon and control sequences that are compatible with the host microorganism can be used as transforming vectors in connection with these hosts. For example, bacteriophage such as lamdaGEM™-11 may be utilized in making a recombinant vector which can be used to transform susceptible host cells such as E. coli LE392.

The expression vector of the invention may comprise two or more promoter-cistron pairs, encoding each of the polypeptide components. A promoter is an untranslated regulatory sequence located upstream (5′) to a cistron that modulates its expression. Prokaryotic promoters typically fall into two classes, inducible and constitutive. Inducible promoter is a promoter that initiates increased levels of transcription of the cistron under its control in response to changes in the culture condition, e.g. the presence or absence of a nutrient or a change in temperature.

A large number of promoters recognized by a variety of potential host cells are well known. The selected promoter can be operably linked to cistron DNA encoding the light or heavy chain by removing the promoter from the source DNA via restriction enzyme digestion and inserting the isolated promoter sequence into the vector of the invention. Both the native promoter sequence and many heterologous promoters may be used to direct amplification and/or expression of the target genes. In some embodiments, heterologous promoters are utilized, as they generally permit greater transcription and higher yields of expressed target gene as compared to the native target polypeptide promoter.

Promoters suitable for use with prokaryotic hosts include the PhoA promoter, the beta-galactamase and lactose promoter systems, a tryptophan (trp) promoter system and hybrid promoters such as the tac or the trc promoter. However, other promoters that are functional in bacteria (such as other known bacterial or phage promoters) are suitable as well. Their nucleotide sequences have been published, thereby enabling a skilled worker operably to ligate them to cistrons encoding the target light and heavy chains (Siebenlist et al. (1980) CELL 20:269) using linkers or adaptors to supply any required restriction sites.

Each cistron within the recombinant vector may comprise a secretion signal sequence component that directs translocation of the expressed polypeptides across a membrane. In general, the signal sequence may be a component of the vector, or it may be a part of the target polypeptide DNA that is inserted into the vector. The signal sequence selected for the purpose of this invention should be one that is recognized and processed (i.e. cleaved by a signal peptidase) by the host cell. For prokaryotic host cells that do not recognize and process the signal sequences native to the heterologous polypeptides, the signal sequence is substituted by a prokaryotic signal sequence selected, for example, from the group consisting of the alkaline phosphatase, penicillinase, Ipp, or heat-stable enterotoxin II (STII) leaders, LamB, PhoE, PelB, OmpA and MBP. In one embodiment of the invention, the signal sequences used in both cistrons of the expression system are STII signal sequences or variants thereof

Alternatively, the antibodies of the invention can be produced in the cytoplasm of the host cell, and therefore does not require the presence of secretion signal sequences within each cistron. In that regard, immunoglobulin light and heavy chains are expressed, folded and assembled to form functional immunoglobulins within the cytoplasm. Certain host strains (e.g., the E. coli trxB-strains) provide cytoplasm conditions that are favorable for disulfide bond formation, thereby permitting proper folding and assembly of expressed protein subunits. Proba and Pluckthun (1995) GENE 159:203.

Antibodies of the invention can also be produced by using an expression system in which the quantitative ratio of expressed polypeptide components can be modulated in order to maximize the yield of secreted and properly assembled antibodies of the invention. Such modulation is accomplished at least in part by simultaneously modulating translational strengths for the polypeptide components.

One technique for modulating translational strength is disclosed in Simmons et al., U.S. Pat. No. 5,840,523. It utilizes variants of the translational initiation region (TIR) within a cistron. For a given TIR, a series of amino acid or nucleic acid sequence variants can be created with a range of translational strengths, thereby providing a convenient means by which to adjust this factor for the desired expression level of the specific chain. TIR variants can be generated by conventional mutagenesis techniques that result in codon changes which can alter the amino acid sequence. In certain embodiments, changes in the nucleotide sequence are silent. Alterations in the TIR can include, for example, alterations in the number or spacing of Shine-Dalgarno sequences, along with alterations in the signal sequence. One method for generating mutant signal sequences is the generation of a “codon bank” at the beginning of a coding sequence that does not change the amino acid sequence of the signal sequence (i.e., the changes are silent). This can be accomplished by changing the third nucleotide position of each codon; additionally, some amino acids, such as leucine, serine, and arginine, have multiple first and second positions that can add complexity in making the bank. This method of mutagenesis is described in detail in Yansura et al. (1992) METHODS: A COMPANION TO METHODS IN ENZYMOL. 4:151-158.

In one embodiment, a set of vectors is generated with a range of TIR strengths for each cistron therein. This limited set provides a comparison of expression levels of each chain as well as the yield of the desired antibody products under various TIR strength combinations. TIR strengths can be determined by quantifying the expression level of a reporter gene as described in detail in U.S. Pat. No. 5,840,523. Based on the translational strength comparison, the desired individual TIRs are selected to be combined in the expression vector constructs of the invention.

Prokaryotic host cells suitable for expressing antibodies of the invention include Archaebacteria and Eubacteria, such as Gram-negative or Gram-positive organisms. Examples of useful bacteria include Escherichia (e.g., E. coli), Bacilli (e.g., B. subtilis), Enterobacteria, Pseudomonas species (e.g., P. aeruginosa), Salmonella typhimurium, Serratia marcescans, Klebsiella, Proteus, Shigella, Rhizobia, Vitreoscilla, or Paracoccus. In one embodiment, gram-negative cells are used. In one embodiment, E. coli cells are used as hosts for the invention. Methods for constructing derivatives of any of the above-mentioned bacteria having defined genotypes are known in the art and described in, for example, Bass et al. (1990) PROTEINS 8:309-314. It is generally necessary to select the appropriate bacteria taking into consideration replicability of the replicon in the cells of a bacterium. For example, E. coli, Serratia, or Salmonella species can be suitably used as the host when well known plasmids such as pBR322, pBR325, pACYC177, or pKN410 are used to supply the replicon. Typically the host cell should secrete minimal amounts of proteolytic enzymes, and additional protease inhibitors may desirably be incorporated in the cell culture.

### Antibody Production

Host cells are transformed with the above-described expression vectors and cultured in conventional nutrient media modified as appropriate for inducing promoters, selecting transformants, or amplifying the genes encoding the desired sequences.

Transformation means introducing DNA into the prokaryotic host so that the DNA is replicable, either as an extrachromosomal element or by chromosomal integrant. Depending on the host cell used, transformation is done using standard techniques appropriate to such cells. The calcium treatment employing calcium chloride is generally used for bacterial cells that contain substantial cell-wall barriers. Another method for transformation employs polyethylene glycol/DMSO. Yet another technique used is electroporation.

Prokaryotic cells used to produce the polypeptides of the invention are grown in media known in the art and suitable for culture of the selected host cells. Examples of suitable media include Luria broth (LB) plus necessary nutrient supplements. In some embodiments, the media also contains a selection agent, chosen based on the construction of the expression vector, to selectively permit growth of prokaryotic cells containing the expression vector. For example, ampicillin is added to media for growth of cells expressing ampicillin resistant gene.

Any necessary supplements besides carbon, nitrogen, and inorganic phosphate sources may also be included at appropriate concentrations introduced alone or as a mixture with another supplement or medium such as a complex nitrogen source. Optionally the culture medium may contain one or more reducing agents selected from the group consisting of glutathione, cysteine, cystamine, thioglycollate, dithioerythritol and dithiothreitol.

The prokaryotic host cells are cultured at suitable temperatures. In certain embodiments, for E. coli growth, growth temperatures range from about 20° C. to about 39° C.; from about 25° C. to about 37° C.; or about 37° C. The pH of the medium may be any pH ranging from about 5 to about 9, depending mainly on the host organism. In certain embodiments, for E. coli, the pH is from about 6.8 to about 7.4, or about 7.0.

If an inducible promoter is used in the expression vector of the invention, protein expression is induced under conditions suitable for the activation of the promoter. In one aspect of the invention, PhoA promoters are used for controlling transcription of the polypeptides. Accordingly, the transformed host cells are cultured in a phosphate-limiting medium for induction. In certain embodiments, the phosphate-limiting medium is the C.R.A.P. medium (see, e.g., Simmons et al. (2002) J. IMMUNOL. METHODS 263:133-147). A variety of other inducers may be used, according to the vector construct employed, as is known in the art.

In one embodiment, the expressed polypeptides of the present invention are secreted into and recovered from the periplasm of the host cells. Protein recovery typically involves disrupting the microorganism, generally by such means as osmotic shock, sonication or lysis. Once cells are disrupted, cell debris or whole cells may be removed by centrifugation or filtration. The proteins may be further purified, for example, by affinity resin chromatography. Alternatively, proteins can be transported into the culture media and isolated therein. Cells may be removed from the culture and the culture supernatant being filtered and concentrated for further purification of the proteins produced. The expressed polypeptides can be further isolated and identified using commonly known methods such as polyacrylamide gel electrophoresis (PAGE) and Western blot assay.

In one aspect of the invention, antibody production is conducted in large quantity by a fermentation process. Various large-scale fed-batch fermentation procedures are available for production of recombinant proteins. Large-scale fermentations have at least 1000 liters of capacity, and in certain embodiments, about 1,000 to 100,000 liters of capacity. These fermentors use agitator impellers to distribute oxygen and nutrients, especially glucose. Small scale fermentation refers generally to fermentation in a fermenter that is no more than approximately 100 liters in volumetric capacity, and can range from about 1 liter to about 100 liters.

In a fermentation process, induction of protein expression is typically initiated after the cells have been grown under suitable conditions to a desired density, e.g., an OD₅₅₀ of about 180-220, at which stage the cells are in the early stationary phase. A variety of inducers may be used, according to the vector construct employed, as is known in the art and described above. Cells may be grown for shorter periods prior to induction. Cells are usually induced for about 12-50 hours, although longer or shorter induction time may be used.

To improve the production yield and quality of the polypeptides of the invention, various fermentation conditions can be modified. For example, to improve the proper assembly and folding of the secreted antibody polypeptides, additional vectors overexpressing chaperone proteins, such as Dsb proteins (DsbA, DsbB, DsbC, DsbD and or DsbG) or FkpA (a peptidylprolyl cis,trans-isomerase with chaperone activity) can be used to co-transform the host prokaryotic cells. The chaperone proteins have been demonstrated to facilitate the proper folding and solubility of heterologous proteins produced in bacterial host cells. Chen et al. (1999) J. BIOL. CHEM. 274:19601-19605; Georgiou et al., U.S. Pat. No. 6,083,715; Georgiou et al., U.S. Pat. No. 6,027,888; Bothmann and Pluckthun (2000) J. BIOL. CHEM. 275:17100-17105; Ramm and Pluckthun (2000) J. BIOL. CHEM. 275:17106-17113; Arie et al. (2001) MOL. MICROBIOL. 39:199-210.

To minimize proteolysis of expressed heterologous proteins (especially those that are proteolytically sensitive), certain host strains deficient for proteolytic enzymes can be used for the present invention. For example, host cell strains may be modified to effect genetic mutation(s) in the genes encoding known bacterial proteases such as Protease III, OmpT, DegP, Tsp, Protease I, Protease Mi, Protease V, Protease VI and combinations thereof. Some E. coli protease-deficient strains are available and described in, for example, Joly et al. (1998), supra; U.S. Pat. Nos. 5,264,365; 5,508,192; Hara et al. (1996) MICROBIAL DRUG RESISTANCE 2:63-72.

In one embodiment, E. coli strains deficient for proteolytic enzymes and transformed with plasmids overexpressing one or more chaperone proteins are used as host cells in the expression system of the invention.

### Antibody Purification

In one embodiment, the antibody protein produced herein is further purified to obtain preparations that are substantially homogeneous for further assays and uses. Standard protein purification methods known in the art can be employed. The following procedures are exemplary of suitable purification procedures: fractionation on immunoaffinity or ion-exchange columns, ethanol precipitation, reverse phase HPLC, chromatography on silica or on a cation-exchange resin such as DEAE, chromatofocusing, SDS-PAGE, ammonium sulfate precipitation, and gel filtration using, for example, Sephadex G-75.

In one aspect, protein A immobilized on a solid phase is used for immunoaffinity purification of the antibody products of the invention. Protein A is a 41 kDa cell wall protein from Staphylococcus aureas which binds with a high affinity to the Fc region of antibodies (Lindmark et al. (1983) J. IMMUNOL. METH. 62:1-13). The solid phase to which Protein A is immobilized can be a column comprising a glass or silica surface, or a controlled pore glass column or a silicic acid column. In some applications, the column is coated with a reagent, such as glycerol, to possibly prevent nonspecific adherence of contaminants.

As the first step of purification, a preparation derived from the cell culture as described above can be applied onto a Protein A immobilized solid phase to allow specific binding of the antibody of interest to Protein A. The solid phase would then be washed to remove contaminants non-specifically bound to the solid phase. Finally the antibody of interest is recovered from the solid phase by elution.

In general, antibodies can be made, for example, using traditional hybridoma techniques (Kohler and Milstein (1975) NATURE 256:495-499), recombinant DNA methods (U.S. Pat. No. 4,816,567), or phage display performed with antibody libraries (Clackson et al. (1991) Nature, 352: 624-628; Marks et al. (1991) J. MOL. BIOL. 222:581-597). For other antibody production techniques, see also ANTIBODIES: A LABORATORY MANUAL, Eds. Harlow et al., Cold Spring Harbor Laboratory, 1988. The invention is not limited to any particular source, species of origin, or method of production.

### Derivatives

This disclosure also provides a method for obtaining an antibody specific for CD33rSiglec. CDRs in such antibodies are not limited to the specific sequences of VH and VL and may include variants of these sequences that retain the ability to specifically bind CD33rSiglec. Such variants may be derived by a skilled artisan using techniques well known in the art. For example, amino acid substitutions, deletions, or additions, can be made in the FRs and/or in the CDRs. While changes in the FRs are usually designed to improve stability and immunogenicity of the antibody, changes in the CDRs are typically designed to increase affinity of the antibody for its target. Variants of FRs also include naturally occurring immunoglobulin allotypes. Such affinity-increasing changes may be determined empirically by routine techniques that involve altering the CDR and testing the affinity antibody for its target. For example, conservative amino acid substitutions can be made within any one of the disclosed CDRs. Various alterations can be made according to the methods described in ANTIBODY ENGINEERING, 2nd ed., Oxford University Press, ed. Borrebaeck, 1995. These include but are not limited to nucleotide sequences that are altered by the substitution of different codons that encode a functionally equivalent amino acid residue within the sequence, thus producing a “silent” change. For example, the nonpolar amino acids include alanine, leucine, isoleucine, valine, proline, phenylalanine, tryptophan, and methionine. The polar neutral amino acids include glycine, serine, threonine, cysteine, tyrosine, asparagine, and glutamine. The positively charged (basic) amino acids include arginine, lysine, and histidine. The negatively charged (acidic) amino acids include aspartic acid and glutamic acid. Substitutes for an amino acid within the sequence may be selected from other members of the class to which the amino acid belongs. Furthermore, any native residue in the polypeptide may also be substituted with alanine (see, e.g., MacLennan et al. (1998) ACTA PHYSIOL. SCAND. SUPPL. 643:55-67; Sasaki et al. (1998) ADV. BIOPHYS. 35:1-24).

Derivatives and analogs of antibodies of the invention can be produced by various techniques well known in the art, for example, as discussed herein above.

### IV. Pharmaceutical Compositions, Methods of Administration, and Therapeutic Uses

The methods and compositions disclosed herein can be used to treat a variety of cancers and cancerous conditions, which include, but are not limited to, epithelial cancers and carcinomas. The term “epithelial cancer” refers to any malignant process that has an epithelial origin. Examples of epithelial cancers include, but are not limited to, a gynecological cancer such as endometrial cancer, ovarian cancer, cervical cancer, vulvar cancer, uterine cancer or fallopian tube cancer, breast cancer, colon cancer, prostate cancer, lung cancer, pancreatic cancer, urinary cancer, bladder cancer, head and neck cancer, oral cancer and liver cancer. An epithelial cancer may be at different stages as well as varying degrees of grading. In embodiments, the epithelial cancer is selected from the group consisting of breast cancer, prostate cancer, lung cancer, pancreatic cancer, bladder cancer and ovarian cancer. In a particular embodiment, the epithelial cancer is breast cancer. In a particular embodiment, the epithelial cancer is ovarian cancer. In a particular embodiment, the epithelial cancer is prostate cancer. In a particular embodiment, the epithelial cancer is lung cancer. In a particular embodiment, the epithelial cancer is head and neck cancer. In a particular embodiment, the epithelial cancer is head and neck squamous cell carcinoma. In a particular embodiment, the epithelial cancer is colon cancer.

The term “carcinoma” refers to a malignant new growth made up of epithelial cells tending to infiltrate the surrounding tissues and give rise to metastases. Exemplary carcinomas which can be treated include, for example, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, baso squamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, and carcinoma villosum.

The terms “subject”, “patient” and “individual” are used interchangeably herein and refer to a warm-blooded animal such as a mammal that is afflicted with, or suspected of having, at risk for or being pre-disposed to, or being screened for cancer, for example, epithelial cancer. The term includes but is not limited to domestic animals, sports animals, primates and humans. Preferably, the terms refer to a human.

A subject suspected of having epithelial cancer includes a subject that presents one or more symptoms indicative of an epithelial cancer (e.g., a noticeable lump or mass) or is being screened for a cancer (e.g., during a routine physical). A subject suspected of having cancer, for example, an epithelial cancer, may also have one or more risk factors. A subject suspected of having epithelial cancer has generally not been tested for cancer. However, a subject suspected of having epithelial cancer encompasses an individual who has received an initial diagnosis but for whom the stage of cancer is not known and people who once had cancer (e.g., an individual in remission).

A subject at risk for or being pre-disposed to cancer, for example, an epithelial cancer, includes a subject with one or more risk factors for developing an epithelial cancer. Risk factors include, but are not limited to, gender, age, genetic predisposition, environmental exposure, previous incidents of cancer, pre-existing non-cancer diseases, and lifestyle.

As used herein, the terms, “treat,” “treatment” and “treating” includes any effect, e.g., lessening, reducing, modulating, ameliorating or eliminating, that results in the improvement of the condition, disease, disorder, and the like, or ameliorating a symptom thereof. The terms “treatment” and “therapeutic method” refer to both therapeutic treatment and prophylactic/preventative measures. Those in need of treatment may include individuals already having a particular medical disorder as well as those who may ultimately acquire the disorder (i.e., those needing preventative measures).

The term “effective amount” refers to a dosage or amount that is sufficient to reduce the activity of a CD33-Like Siglec to result in amelioration of symptoms in a patient or to achieve a desired biological outcome, e.g., inhibit the binding of the natural Siglec ligand to the Siglec, blocking the ability of the Siglec to bind a sialoglycoprotein, suppressing the innate or adaptive immune response, blocking the ability of Siglecs to recruit inhibitory proteins such as SHP phosphatases via their ITIM domains.

The disclosure provides compositions comprising anti-CD33rSiglec antibodies. Such compositions may be suitable for pharmaceutical use and administration to patients. The compositions typically comprise one or more antibodies of the present invention and a pharmaceutically acceptable excipient. The phrase “pharmaceutically acceptable excipient” includes any and all solvents, dispersion media, coatings, antibacterial agents and antifungal agents, isotonic agents, and absorption delaying agents, and the like, that are compatible with pharmaceutical administration. The use of such media and agents for pharmaceutically active substances is well known in the art. The compositions may also contain other active compounds providing supplemental, additional, or enhanced therapeutic functions. The pharmaceutical compositions may also be included in a container, pack, or dispenser together with instructions for administration.

A pharmaceutical composition of the invention is formulated to be compatible with its intended route of administration. Methods to accomplish the administration are known to those of ordinary skill in the art. The administration may, for example, be intravenous, intraperitoneal, intramuscular, intracavity, subcutaneous or transdermal. It may also be possible to obtain compositions which may be topically or orally administered, or which may be capable of transmission across mucous membranes.

Solutions or suspensions used for intradermal or subcutaneous application typically include one or more of the following components: a sterile diluent such as water for injection, saline solution, fixed oils, polyethylene glycols, glycerin, propylene glycol, or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates; and agents for the adjustment of tonicity such as sodium chloride or dextrose. The pH can be adjusted with acids or bases, such as hydrochloric acid or sodium hydroxide. Such preparations may be enclosed in ampoules, disposable syringes or multiple dose vials made of glass or plastic.

Pharmaceutical compositions suitable for injection include sterile aqueous solutions or dispersions and sterile powders for the extemporaneous preparation of sterile injectable solutions or dispersion. For intravenous administration, suitable carriers include physiological saline, bacteriostatic water, Cremophor EL (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars; polyalcohols such as mannitol, sorbitol, and sodium chloride in the composition. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and/or by the use of surfactants. Prolonged absorption of the injectable compositions can be brought about by including in the composition an agent which delays absorption, for example, aluminum monostearate, and gelatin.

Oral compositions generally include an inert diluent or an edible carrier. They can be enclosed in gelatin capsules or compressed into tablets. For oral administration, the antibodies can be combined with excipients and used in the form of tablets, troches, or capsules. Pharmaceutically compatible binding agents, and/or adjuvant materials can be included as part of the composition. The tablets, pills, capsules, troches, and the like can contain any of the following ingredients, or compounds of a similar nature; a binder such as microcrystalline cellulose, gum tragacanth or gelatin; an excipient such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

Systemic administration can also be by transmucosal or transdermal means. For transmucosal or transdermal administration, penetrants appropriate to the barrier to be permeated are used in the formulation. Such penetrants are generally known in the art, and include, for example, detergents, bile salts, and fusidic acid derivatives. Transmucosal administration may be accomplished, for example, through the use of lozenges, nasal sprays, inhalers, or suppositories. For example, in case of antibodies that comprise the Fc portion, compositions may be capable of transmission across mucous membranes in intestine, mouth, or lungs (e.g., via the FcRn receptor-mediated pathway as described in U.S. Pat. No. 6,030,613). For transdermal administration, the active compounds may be formulated into ointments, salves, gels, or creams as generally known in the art. For administration by inhalation, the antibodies may be delivered in the form of an aerosol spray from pressured container or dispenser, which contains a suitable propellant, e.g., a gas such as carbon dioxide, or a nebulizer.

In certain embodiments, the presently disclosed antibodies are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. Liposomal suspensions containing the presently disclosed antibodies can also be used as pharmaceutically acceptable carriers. These can be prepared according to methods known to those skilled in the art, for example, as described in U.S. Pat. No. 4,522,811.

It may be advantageous to formulate oral or parenteral compositions in a dosage unit form for ease of administration and uniformity of dosage. The term “dosage unit form” as used herein refers to physically discrete units suited as unitary dosages for the subject to be treated; each unit containing a predetermined quantity of active compound calculated to produce the desired therapeutic effect in association with the required pharmaceutical carrier.

Toxicity and therapeutic efficacy of the composition of the invention can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compositions that exhibit large therapeutic indices are preferred.

For any composition used in the present invention, the therapeutically effective dose can be estimated initially from cell culture assays. Examples of suitable bioassays include DNA replication assays, cytokine release assays, transcription-based assays, CD33rSiglec binding assays, IMIT based assays, immunological assays, and other assays as, for example, described in the Examples. The data obtained from the cell culture assays and animal studies can be used in formulating a range of dosage for use in humans. A dose may be formulated in animal models to achieve a circulating plasma concentration range that includes the IC50 (i.e., the concentration of the antibody which achieves a half-maximal inhibition of symptoms). Circulating levels in plasma may be measured, for example, by high performance liquid chromatography. The effects of any particular dosage can be monitored by a suitable bioassay. The dosage lies preferably within a range of circulating concentrations with little or no toxicity. The dosage may vary depending upon the dosage form employed and the route of administration utilized.

Mouse models with humanized immune systems represent useful platforms to evaluate compounds to treat a variety of human diseases, from cancer and infectious diseases to allergies, inflammation and Graft versus Host Disease. JAX humanized NSG™ mice generate functional human immune systems with different capabilities.

A humanized mouse is a mouse carrying functioning human genes, cells, tissues, and/or organs. Humanized mice are commonly used as small animal models in biological and medical research for human therapeutics. Immunodeficient mice are often used as recipients for human cells or tissues, because they can relatively easily accept heterologous cells due to lack of host immunity. Traditionally, the nude mouse and severe combined immunodeficiency (SCID) mouse have been used for this purpose, but recently the NOG mouse and the NSG mouse have been shown to engraft human cells and tissues more efficiently than other models. Two mouse strains, called MITRG and MISTRG, were described in which human versions of four genes encoding cytokines important for innate immune cell development are knocked into their respective mouse loci. Such humanized mouse models may be used to model the human immune system in scenarios of health and pathology, and may enable evaluation of therapeutic candidates in an in vivo setting relevant to human physiology.

Humanized CD34+ mice are in vivo models to study immuno-oncology, infectious diseases and graft rejection research. This model has the longest research span, over 12 months with a functional human immune system and displays T-cell dependent inflammatory responses, with no donor cell immune reactivity towards host. Hu-CD34+ mice are produced by injecting CD34+ and yield robust multilineage immune systems with good T cell maturation and function for long-term studies.

Humanized PBMC mice are used as in vivo models to study and evaluate compounds for infectious diseases and graft rejection research. This model has the fastest engraftment rate using adult peripheral blood mononuclear cells and enables short-term studies requiring a strong effector and memory T cell function.

Throughout the description, where compositions and kits are described as having, including, or comprising specific components, or where processes and methods are described as having, including, or comprising specific steps, it is contemplated that, additionally, there are compositions and kits of the present invention that consist essentially of, or consist of, the recited components, and that there are processes and methods according to the present invention that consist essentially of, or consist of, the recited processing and method steps.

Certain numerical values presented herein are preceded by the term “about.” The term “about” is used to provide literal support for the numerical value the term “about” precedes, as well as a numerical value that is approximately the numerical value, that is the approximating unrecited numerical value may be a number which, in the context it is presented, is the substantial equivalent of the specifically recited numerical value.

When a range of numerical values is presented herein, it is contemplated that each intervening value between the lower and upper limit of the range, the values that are the upper and lower limits of the range, and all stated values with the range are encompassed within the disclosure. All the possible sub-ranges within the lower and upper limits of the range are also contemplated by the disclosure.

### EXAMPLES

The invention will be more fully understood by reference to the following examples, which provide illustrative non-limiting embodiments of the invention. The examples describe the use of antibodies to Siglecs, and in particular CD33 related Siglecs, and their ability to block or interfere with ligand binding. Preferred antibodies inhibit the binding of the natural Siglec ligand to the Siglec, resulting in interfering with the normal suppression or inhibition of innate or adaptive immunity. Binding of preferred antibodies blocks the ability of the Siglec to bind a sialoglycoprotein, blocks the ability of the Siglec to suppress the innate or adaptive immune response, and/or blocks the ability of Siglecs to recruit inhibitory proteins such as SHP phosphatases via their ITIM domains.

### Example 1—Anti-Siglec Antibodies

This Example describes a number of assays that can be used to identify anti-Siglec antibodies useful in the practice of the invention.

### A. Siglec-3 Assay

Tyrosine phosphorylation of the two intra-cellular tyrosine-based motifs of Siglec-3 by ligand binding to the receptor or by treatment with the protein tyrosine phosphatase inhibitor pervanadate results in the recruitment of several SH2 domain-containing proteins such as SHP-1 and SHP-2, Syk, CrkL, and PLC-γl. Based on mutagenesis studies, the membrane-proximal ITIM motif appears to be dominant in Siglec-3 interactions with the inhibitory tyrosine phosphatases SHP-1 and SHP-2. CD33 tyrosine phosphorylation is dependent on Src family kinases, and the Src kinase Lck are effective at phosphorylating the proximal, but not the distal, tyrosine residue of human Siglec-3. It is contemplated that anti-Siglec-3 antibodies useful in the practice of the invention will block ligand binding or block ligand dependent phosphorylation or block SHP-1 and/or SHP-2 association.

### B. Siglec-5 Assay

Siglec-5 can recruit SHP-1 and SHP-2 after tyrosine phosphorylation and inhibit calcium flux and serotonin release after co-ligation with the ITAM-containing high-affinity IgE receptor (FIERI) (Avril et al. (2005) J. BIOL. CHEM. 280:19843-198451). It is contemplated that anti-Siglec-5 antibodies useful in the practice of the invention will block ligand binding or block ligand dependent phosphorylation.

### C. Siglec-6 Assay

As shown in Lam et al. (2011) J. BIOL. CHEM. 286(43):37118-27, Glycodelin-A protein interacts with Siglec-6 protein to suppress trophoblast invasiveness by down-regulating extracellular signal-regulated kinase (ERK)/c-Jun signaling pathway. The binding of GdA to Siglec-6 was sialic acid-dependent. It is contemplated that anti-Siglec-6 antibodies useful in the practice of the invention will block ligand binding or block ligand dependent phosphorylation.

### D. Siglec-7 Assay

Using standard killing assays, the inhibitory effects of Siglec-7 on natural killer (NK)-mediated cytotoxicity can be observed in cell lines and primary human cells (Falco et al. (1999) J. EXP. MED. 190:793-802; Nicoll et al. (1999) JBC 274:34089-34095). Siglec-7 binds to the ganglioside GD3, which displays α2-8-linked disialic acids. It is contemplated that anti-Siglec-7 antibodies useful in the practice of the invention will block ligand binding or block ligand dependent phosphorylation or inhibit ligand dependent functions as described above.

### E. Siglec-8 Assay

Siglec-8 ligation with monoclonal antibodies, as well as autoantibodies contained in commercial gamma globulin (IVIg) preparations, has been used to explore biological function. With respect to eosinophils, this results in caspase-, mitochondrial-, and reactive oxygen species-dependent apoptosis (Nutku et al. (2003) BLOOD 101:5014-520; Von Guten et al. (2007) J. ALLERGY CLIN. IMMUNOL. 119:1005-1011). Although little is known about Siglec-8 function on basophils, it is now known that Siglec-8 engagement on mast cells does not affect their survival but instead counteracts stimulatory signals delivered via FcεRI crosslinking, resulting in 50% or greater inhibition of release of histamine and prostaglandin D2 but not release of cytokines such as IL-8 (Yokoi et al. (2008) J. ALLERGY CLIN. IMMUNOL. 121:499-505). Siglec-8 engagement was also shown to inhibit mast cell calcium flux associated with FcεRI activation, and this inhibitory effect was lost in Siglec-8-transfected RBL cells when a point mutation was introduced into the membrane-proximal ITIM domain of Siglec-8. It is contemplated that anti-Siglec-8 antibodies useful in the practice of the invention will block ligand binding or block ligand dependent phosphorylation or inhibit ligand dependent functions as described above.

### F. Siglec-9 Assay

One useful assay is based on the observation that ligation of Siglec-9 by antibodies induced apoptosis in human neutrophils (Von Guten et al. (2005) BLOOD 106:1423-1431). Siglec-9-mediated cell death was enhanced by cytokines such as granulocyte/macrophage colony-stimulating factor (GM-CSF), interferon-α (IFN-α), and IFN-γ, and “primed” neutrophils from patients with sepsis or rheumatoid arthritis were more susceptible to Siglec-9-mediated death than normal cells. Incubation with GM-CSF can result in rapid tyrosine phosphorylation of Siglec-9 and significantly increase the potency and efficacy of Siglec-9-dependent death upon antibody crosslinking. It is contemplated that anti-Siglec-9 antibodies useful in the practice of the invention will block ligand binding or block ligand dependent phosphorylation or inhibit ligand dependent functions as described above.

### G. Siglec-10 Assay

Siglec-10 (also referred to as Siglec-like gene (SLG2) is expressed on monocytes, dendritic cells, a CD16+/CD56-subpopulation of cells, and weakly on eosinophils and B cells. Like other family members, Siglec-10 mediates sialic acid-dependent binding of transfected COS cells to human erythrocytes and binding is masked by endogenous ligands in cis on these cells (Munday et al. (2001) BIOCHEM. J. 355:489-497). Sialoconjugates in α2-3 and α2-6 linkage are recognized by Siglec-10, with a preference for the latter. In kinase assays, Siglec-10 was phosphorylated in decreasing order by Lck, Jak3, and Emt, but not ZAP-70 (Whitney et al. (2001) EUR. J. BIOCHEM. 268:6083-6096). SHP-1 and SHP-2 have been shown to associate with Siglec-10, whereas no interaction with the SH2-protein SAP (SLAM-associated protein) was observed (Kitzig et al. (2002) BBRC 296:355-362). It is contemplated that anti-Siglec-10 antibodies useful in the practice of the invention will block ligand binding or block ligand dependent phosphorylation or block SHP-1 and/or SHP-2 association.

### H. Siglec-11 Assay

Analogous to Siglec-10, tyrosine-phosphorylation of Siglec-11 following ligand binding leads to recruitment of SHP-1 and SHP-2 as is typical for CD33-related Siglecs. It is contemplated that anti-Siglec-11 antibodies useful in the practice of the invention will block ligand binding or block ligand dependent phosphorylation or block SHP-1 and/or SHP-2 association.

Proteome Profiler™ Human Phospho-Immunoreceptor Array Kit (R&D Systems; ARY004B) is a membrane-based antibody array for the parallel determination of the relative phosphorylation of human immunoreceptors, including Siglec 3, 5, 7, 9 and 10. It is contemplated that anti-Siglec-3, anti-Siglec-5, anti-Siglec-7, anti-Siglec-9, and/or anti-Siglec-10, antibodies useful in the practice of the invention will block ligand binding or block ligand dependent phosphorylation in this assay.

### Example 2—Anti-Siglec Antibody Assays

Antibodies useful in the practice of the invention may be determined using the additional assays described in this Example.

### A. Ligand Blocking Assays

Antibodies useful in the practice of the invention can be assayed for their ability to block CD33-like Siglec binding to erythrocytes. Erythrocyte binding assays are performed using stable CHO cell lines independently expressing each CD33-like Siglec. CHO cells stably expressing various CD33-like Siglecs are generated by transfection with full-length Siglec cDNA cloned, for instance, into the pcDNA3 vector (Invitrogen). G418-resistant CHO cell clones expressing Siglecs are identified by their ability to bind anti-Siglec mAbs. Erythrocytes or red blood cells (RBCs) are obtained from human blood from healthy volunteers. After centrifugation at 500 g, the RBCs are washed 3 times with PBS buffer. Then, 10 μl of compacted RBCs are resuspended with 490 μl of 0.05 M HEPES buffer and added to the CHO cells, which have optionally been pretreated with 10 mU of neuraminidase (Sigma, St. Louis, Mo., USA) for 30 minutes at 37° C. After incubation, the unbound RBCs are removed with 3 PBS washes, and the remaining bound RBCs are lysed with water to release hemoglobin possessing pseudoperoxidase activity. Lysates are diluted into citrate buffer containing urea, hydrogen peroxidase, and o-phenylenediamine dihydrochloride and incubated at room temperature for 0.5 hours before absorbance at 504 nm is measured. Alternatively, antibodies can be tested for their ability to neutralize, reduce or block CD33-like Siglec-mediated adhesion of human red blood cells to recombinant human Siglec Fc chimeras as described by Kelm, S. et al. (1994) CURRENT BIOLOGY 4:965. It is contemplated that anti-Siglec antibodies useful in practice of the invention, e.g., anti-Siglec-7 and/or anti-Siglec-9 antibodies, will reduce or block the ability of the erythrocytes to bind to the CD33-like Siglec.

Antibodies useful in the practice of the invention can be assayed for their ability to block CD33-like Siglec binding to polyacrylamide (PAA) glycoconjugates. CHO cells stably expressing various CD33-like Siglecs can be used for binding assays with biotinylated PAA glycoconjugates (2,3-PAA, NeuAca2,3Galb1,4Glc coupled to PAA; 2,6-PAA, NeuAca2,6Galb1,4Glc coupled to PAA) as described previously (Nicoll, G. et al. (1999) J. BIOL. CHEM. 274, 34089-34095). Briefly, Siglec expressing-CHO cells or wild-type CHO cells are optionally pretreated with 10 mU of neuraminidase (Sigma, St. Louis, Mo., USA) for 30 minutes at 37° C. to remove cell surface sialic acids and then incubated with saturating concentrations (20 mg/ml) of various PAA conjugates. After 1 hour, cells are washed and binding of PAA conjugates is detected by incubation with fluorescein-streptavidin. For ligand displacement, antibodies selective for a particular Siglec are incubated either ahead of PAA glycoconjugate incubation (for instance 30 or 60 minutes at 4° C. prior) or at the same time. It is contemplated that anti-Siglec antibodies useful in practice of the invention, e.g., anti-Siglec-7 and/or anti-Siglec-9 antibodies, will reduce or block the ability of the PAA conjugates to bind to the CD33-like Siglec.

Antibodies useful in the practice of the invention can be assayed for their ability to block CD33-like Siglec binding to other representative CD33-like Siglec ligands, including MUC1-ST, CA15.3, CA125, MUC16, human erythrocytes, glycophorin A, osteopontin, hyaluronan, LGALS3BP, and cancer cell lines that express Siglec ligands. For binding assays using MUC1-ST, MUC1-ST is prepared as follows. Recombinant secreted MUC1 consisting of 16 tandem repeats carrying sialylated core 1 and fused to mouse Ig is produced in CHO cells as described in Backstrom et al. (2003) BIOCHEM. J. 376, 677-686. Concentrated supernatant is treated with 10 mg trypsin per mg MUC1-ST-IgG for 2 hours (MUC1 tandem repeats are not sensitive to trypsin digestion) to remove the Ig. The treated supernatant is applied to a HiPrep 16/10 Q FF anion exchange column, which is washed to remove the unbound material with 20 column volumes of 50 mM Tris-HC1 pH 8.0. The MUC1-ST is eluted as described in Backstrom et al., supra.

### B. Cellular Activation Assays

The ability of Siglec antibodies of the present invention to modulate Siglec-mediated receptor activation in the presence of ligand can be tested in cells engineered to express Siglec proteins, immune cell lines that endogenously express Siglec proteins, or primary immune cells purified from human whole blood. Such assays can be conducted using CHO cells expressing Siglec as described in Zhang et al. (2000) JOURNAL OF BIOLOGICAL CHEMISTRY 271(29):22121, differentiated THP-1 immune cell line as described in Li et al. (2015) NATURE: SCIENTIFIC REPORTS 6:21044, and primary human monocytes, macrophages, neutrophils, NK cells, dendritic cells, and T-cells and as described below. To assay the effects of antibodies on early signaling events, cells are incubated with Siglec ligand in the presence or absence of antibody for 0-30 minutes and the resulting activation of Siglec receptor phosphorylation, SHP-1/2 phosphorylation, calcium flux, and other signal transduction molecules such as phosphorylated Erk1/2 and MEK1/2 (Beatson et al. (2016) NATURE IMMUNOLOGY 17(11):1273) can be determined. To measure phosphorylated proteins, antibodies to Siglecs or SHP-1/2 (for example, those included in the Proteome Profiler Human Phospho-Immunoreceptor Array Kit, R&D Systems), Erk1/2, or MEK1/2 (Cell Signaling Technologies) are plated overnight on plastic before being blocked with 1% BSA in PBS. Clarified supernatants of lysed cells incubated with ligand in the presence or absence of antibody are added and incubated for 2 hours. After incubation with 1 μg/ml biotinylated antibody to phosphorylated tyrosine (for the detection of phosphorylated SHP-1, SHP-2 or Siglecs) or phospho-specific antibodies (for the detection of phosphorylated Erk1/2 or MEK1/2), streptavidin-HRP and substrate are added and OD₄₅₀ is measured. To measure calcium flux, cells pre-labeled with an intracellular calcium reporter (Fluo-4; Life Technologies) are treated with Siglec ligand for 4 hours at 4° C., with or without anti-Siglec antibodies of the present invention. The cells are brought up to 37° C. and calcium flux is measured at 530 nm using a plate reader. It is contemplated that anti-Siglec antibodies useful in practice of the invention, e.g., anti-Siglec-7 and/or anti-Siglec-9 antibodies, will antagonize the effects of Siglec ligands in these assays.

### C. Monocyte/Macrophage Assays

Isolated human primary monocytes or monocyte-derived macrophages are used to evaluate whether preferred antibodies of the present invention will reduce or block the ability of ligands (e.g., MUC1-ST) to bind to the CD33-like Siglec and reduce or block ligand dependent production of cytokines and cancer progression factors. To differentiate monocytes into macrophages, CD14+ cells are plated at a concentration of 1×10⁶/ml in AIM V medium (Lonza) with either 50 ng/ml recombinant human M-CSF, 50 ng/ml recombinant human GM-CSF (R&D Systems), or MUC1-ST. The cytokines are added every 3 days. The cells are incubated at 37° C., 5% CO₂ for 7 days to fully differentiate, before being characterized as macrophages via phenotypic flow cytometric analysis. For binding assays, 1×10⁵ isolated monocytes/differentiated cells at 5×10⁵ cells per ml are incubated for 4 hours on ice with 10 μg of biotinylated recombinant MUC1-ST in 0.5% BSA in PBS. Cells are washed in 0.5% BSA in PBS before 1:200 SAPE (Life Technologies) is added for 30 minutes on ice. Cells are washed and analyzed by flow cytometry or fluorescent microscopy (after cytospin). For cytokine and progression factor assays, isolated monocytes or monocyte-derived macrophages are treated with 100 μg/10⁶ cells MUC1-ST for 4 hours at 4° C., washed, and incubated at 37° C. for 48 hours in AIM-V serum-free media followed by ELISA analysis of interleukin-6 (IL-6), macrophage-stimulating cytokine M-CSF, and plasminogen-activator inhibitor PAI-1. For differentiation assays, monocytes are incubated with MUC1-ST for 4 hours at 4° C. and then differentiated in the presence of M-CSF. Preferred antibodies of the present invention will block or reduce MUC1-ST binding and secretion of the aforementioned factors in monocytes and macrophages as well as induction of immune cell markers of macrophage differentiation such as CD206, CD163, and PD-L1.

### D. Human Neutrophil Assays

Antibodies useful in the practice of the invention can be assayed for their ability to affect tumor cell apoptosis mediated by human neutrophils. Human neutrophils are prepared by separation from peripheral blood mononuclear cells (PBMCs) via Ficoll gradient and subsequent dextran sedimentation. Tumor cell apoptosis is assayed using fluorescently labeled tumor cells and by intracellular staining of cleaved caspase 3 (Cell Signaling) by flow cytometry. For example, MC38GFP cells or CFSE pre-labeled LS180 are seeded on a 96 well plate overnight and neutrophils are added at effector to target ratios at approximately 20:1 or 40:1. Fluorescent cells are then quantified at different time points. Analysis of ligand induced extracellular ROS production can also be analyzed using the OxyBurst assay (Invitrogen). It is contemplated that anti-Siglec antibodies useful in practice of the invention, e.g., anti-Siglec-7 and/or anti-Siglec-9 antibodies, will increase neutrophil killing of target tumor cells.

Antibodies useful in the practice of the invention can be assayed for their ability to effect ligand mediated SHP-1 recruitment to Siglecs in human neutrophils. Freshly isolated human neutrophils are co-cultured for 30 minutes on LS180 or A549 tumor cells or on empty culture dishes. Non-adherent neutrophils are washed away and cells are lysed directly on culture dishes in buffer containing 1% Nonidet-P40 and 1:50 protease inhibitor cocktail (Calbiochem), PhosStop (Roche) and micrococcal nuclease. CD33-like Siglecs are bound with specific Siglec detection antibodies overnight at 4° C. Secondary biotinylated antibody and streptavidin-sepharose beads (GE Healthcare) are used to precipitate CD33-like Siglecs. Immunoprecipitates are loaded on a 10% polyacrylamide gel and blotted on a PVDF membrane. SHP-1 is detected with polyclonal anti-rabbit antibody (Santa Cruz). Blocking and antibody incubation conditions are conducted in 1× Dulbecco's phosphate buffered saline with 0.05% Tween-20 (PBST). Blots are blocked in 5% BSA/PBST and probed with anti-SHP-1/SH-PTP-1 mAb (1:500 dilution) followed by goat anti-rabbit IgG-FITC (1:3,000) in 3% BSA/PBST. Tyrosine phosphorylation was probed with anti-phosphotyrosine-HRP mAb (1:2,000) in 6% BSA/PBST. It is contemplated that anti-Siglec antibodies useful in practice of the invention, e.g., anti-Siglec-7 and/or anti-Siglec-9 antibodies, will block or reduce SHP-1 recruitment to the CD33-like Siglec ITIM domain when co-cultured with tumor cells.

### E. Cell Cytotoxicity Assays

Antibodies useful in the practice of the invention can be assayed for their ability to affect PBMC or NK cell mediated antibody-dependent cellular cytotoxicity (ADCC). ADCC is analyzed by measuring lactate dehydrogenase (LDH) release from breast cancer cells as a result of ADCC activity of PBMCs or NK cells. Tumor cells (target cells) are co-incubated with PBMCs or NK cells (effector cells) at various effector/target (E/T) ratios in the presence or absence of antibodies to CD33-like Siglecs in triplicate. In a typical experiment, 100 μL of PBMCs or NK cells are added to a V-bottom 96-well plate containing 100 μL of target cells at 2×10⁵ cells/mL. After 4 hours, supernatants are collected, and LDH release is measured using a LDH cytotoxicity assay kit (Thermo Fisher Scientific, 88954) according to the manufacturer's protocol. The absorbance at 490 nm is measured with a SpectraMax i3x (Molecular Devices). Specific lysis is calculated as 100×(experimental−effector cells spontaneous release−target cells spontaneous release)/(target cells maximum release−target cells spontaneous release). It is contemplated that anti-Siglec antibodies useful in practice of the invention, e.g., anti-Siglec-7 and/or anti-Siglec-9 antibodies, will increase ADCC activity.

### F. Human T-cell Assays

The ability of antibodies useful in the practice of the invention to modulate human T-cell activity can be tested in activated human T-cell lines engineered to express Siglec proteins or primary T-cells in the presence of Siglec ligands or cancer cells. Such assays can be performed using Jurkat cells stably expressing a specific Siglec receptor and an NFAT reporter gene as described in Ikehara et al. (2004) JOURNAL OF BIOLOGICAL CHEMISTRY 279 (41):43117. Siglec expressing Jurkat cell lines with an NFAT reporter can be generated via stable incorporation of Siglec into the cellular genome of Jurkat-Lucia™ NFAT Cells (Invivogen, Inc.) using lentiviral particles encoding a human Siglec (Origene, Inc.). Jurkat-derived cell lines (1×10⁷) are incubated with anti-Siglec antibodies, Siglec ligand and/or cancer cells plus anti-CD3 and anti-CD28 antibodies (5 μg/ml) or PHA (1.25 μg/ml) in complete RPMI media for 24-48 hours for Jurkat cells at 37° C., 5% CO2. Clarified supernatants of lysed Jurkat cells are then collected and assayed for luciferase activity with a commercially available kit (Invivogen) and IL-2 by ELISA (R&D Systems, Inc.) in a plate reader (i3x instrument, Molecular Devices) according to the manufacturer's instructions. It is contemplated that anti-Siglec antibodies useful in practice of the invention, e.g., anti-Siglec-7 and/or anti-Siglec-9 antibodies, will block or reduce ligand induced NFAT activation.

Assays may also be performed using primary T-cells purified from the whole blood of cancer patients. CD8+ cells are isolated from human PBMCs using microbeads (MACS system; Miltenyi Biotech) according to the manufacturer's instructions. CD8+ T cells (mixed-lymphocyte reaction) labeled with eFluor 670 (eBioscience) at a 1:1 ratio are co-incubated with Siglec antibodies in the presence of anti-CD3/CD28 beads. The proliferation of CD8+ T cells, cell-surface expression of CD69 and CD25 and production of IFN-γ are measured by flow cytometry as described by Beatson et al. (2016) NATURE IMMUNOLOGY 17(11):1273. It is contemplated that anti-Siglec antibodies useful in practice of the invention, e.g., anti-Siglec-7 and/or anti-Siglec-9 antibodies, will block or reduce ligand induced expression of CD69, CD25 or IFN-γ.

### G. Antibody and Receptor Internalization Assays

Antibodies useful in the practice of the invention can be assayed for their ability to effect Siglec endocytosis and receptor internalization. To measure cellular internalization of preferred antibodies of the current invention and receptor endocytosis, cells engineered to express Siglec proteins, immune cell lines that endogenously express Siglec proteins, or primary immune cells purified from human whole blood can be used. Cells previously treated with and without neuraminidase (10⁷ cells/mL in 0.25% BSA/2 mM CaCl/DMEM with 0.1 U/mL Vibrio cholerae sialidase for 1 hour at 37° C., and washed twice in DMEM/F12 and HBSS) are incubated with preferred Siglec antibodies in the presence and absence of Siglec ligand at 4° C. for 1 hour. Labeled cells are washed to remove unbound antibodies and ligands and incubated at 37° C. for 0 to 1 hour. Cells are washed, fixed, and permeabilized, and stained with fluorescein isothiocyanate (FITC)-labeled Siglec antibody (e.g. clone E10-286, available from BD Biosciences specific to Siglec-9 or clone Z176, available from Biolegend specific to Siglec-7), a non-ligand competing antibody that recognizes Siglec protein, and a DyLight 594-labeled isotype-matched secondary antibody. Antibodies and Siglec receptors are visualized by fluorescence microscopy. Images are captured in confocal mode using an Olympus IX81 fluorescence microscope equipped with a dry 60×objective and×2 optical zoom. Acquired images are processed using CellSens Dimension image-processing software (Olympus Life Science) as described in Ortiz et al. (2016) SCI. TRANSL. MED. 8, 365ra158. It is contemplated that anti-Siglec antibodies useful in practice of the invention, e.g., anti-Siglec-7 and/or anti-Siglec-9 antibodies, will block or reduce ligand induced crosslinking, ligation, clustering or endocytosis of Siglecs.

Alternatively, antibody or receptor internalization can be as follows. Neuraminidase-treated cells are incubated with preferred antibodies in the presence and absence of Siglec ligand at 37° C. for different time points. At each time point, cells are washed twice with ice-cold PBS and fixed with 4% PFA in PBS solution (Thermo Fisher) for 15 minutes on ice. Fixed cells are washed twice with PBS, incubated with non-ligand competing Siglec antibody (as described above) and/or a FITC-labeled isotype-matched secondary antibody in PBS for 20 minutes, subsequently washed twice with PBS, and resuspended in 100 ml of fluorescence-activated cell sorting (FACS) buffer (phosphate-buffered saline (PBS) with 2% fetal bovine serum). Trypan blue is added to quench the cell surface FITC signal, and samples are read on a BD Celesta flow cytometer. It is contemplated that anti-Siglec antibodies useful in practice of the invention, e.g., anti-Siglec-7 and/or anti-Siglec-9 antibodies, will block or reduce ligand induced crosslinking, ligation, clustering or endocytosis of Siglecs.

### Example 3—Anti-Siglec-9 Antibodies

Anti-Siglec-9 antibodies useful in the practice of the invention can be identified using the combination of assays described in this Example.

Anti-Siglec-9 antibodies useful in the practice of the invention can be generated and evaluated as follows. Crude supernatant from hybridoma clones expressing anti-Siglec antibodies are assayed for binding to human and cyno Siglec-9 by ELISA or FACS using Siglec-9 expressing CHO cells. Hybridoma clones are also assayed for binding against other CD-33 like Siglecs, e.g., Siglec-3, -5, -6, -7, -8, -10 and/or -11, to ensure antibody specific for Siglec-9 over other CD-33 like Siglecs. Binding affinity is determined and epitope mapping is carried out for hybridoma clones of interest.

Antibodies from hybridoma clones that exhibit desired properties, including Siglec-9 binding and specificity, can be purified and assayed for binding to human and cyno Siglec-9 using immune cells lines or primary cells. Purified antibodies can be assayed for their ability to block or displace binding of Siglec-9 ligands, including erythrocytes, PAA glycoconjugates and MUC1-ST, as described in Example 2.

Purified antibodies that exhibit desired properties, including inhibition of erythrocyte, PAA glycoconjugate and/or MUC1-ST binding to Siglec-9, can be tested for their ability to inhibit receptor activation, including Siglec receptor, SHP-1/2, Erk1/2 , and MEK phosphorylation, and calcium flux as described in Example 2. Purified antibodies can further be tested for internalization and endocytosis as described in Example 2.

Purified antibodies that exhibit desired properties, including inhibition of Siglec receptor, SHP-1/2, Erk1/2, and MEK phosphorylation, and calcium flux and inhibition of Siglec endocytosis, can be tested in macrophage differentiation, neutrophil killing, or NK killing assays, as described in Example 2.

Purified antibodies that exhibit desired properties, including inhibition of macrophage differentiation, promotion of neutrophil killing of tumor cells, and promotion of NK killing of tumor cells can be sequenced, cloned, and expressed for additional assays. Antibodies may be optimized by mutagenesis or affinity maturation.

### Example 4—Testing of Anti-Siglec Antibodies in Animal Models

Numerous murine models have been developed to study human cancer. These models are used to investigate the factors involved in malignant transformation, invasion and metastasis, as well as to examine response to therapy. One of the most widely used models is the human tumor xenograft. In this model, human tumor cells are transplanted, either under the skin or into the organ type in which the tumor originated, into immunocompromised mice that do not reject human cells. For example, the xenograft will be readily accepted by athymic nude mice, severely compromised immunodeficient (SCID) mice, or other immunocompromised mice.

To evaluate the use of Siglec antibodies of the present invention, mouse models with humanized immune systems are used. A humanized mouse is a mouse carrying functioning human genes, cells, tissues, and/or organs. Humanized mice are commonly used as small animal models in biological and medical research for human therapeutics. Immunodeficient mice are often used as recipients for human cells or tissues, because they can relatively easily accept heterologous cells due to lack of host immunity. Traditionally, the nude mouse and severe combined immunodeficiency (SCID) mouse have been used for this purpose, but recently the NOG mouse and the NSG mouse have been shown to engraft human cells and tissues more efficiently than other models. Two mouse strains, called MITRG and MISTRG have been developed in which human versions of four genes encoding cytokines important for innate immune cell development are knocked into their respective mouse loci. Such humanized mouse models may be used to model the human immune system in scenarios of health and pathology, and may enable evaluation of therapeutic candidates in an in vivo setting relevant to human physiology.

Humanized CD34+ mice are good in vivo models for studying immunooncology, infectious diseases and graft rejection research. This model has the longest research span, over 12 months, with a functional human immune system and displays T-cell dependent inflammatory responses, with no donor cell immune reactivity towards host. Hu-CD34+ mice are produced by injecting CD34+ and yield robust multilineage immune systems with good T cell maturation and function for long-term studies.

Humanized PBMC mice are used as in vivo models to study and evaluate compounds for infectious diseases and graft rejection research. This model has the fastest engraftment rate using adult peripheral blood mononuclear cells and enables short-term studies requiring a strong effector and memory T cell function.

It is contemplated that preferred antibodies to CD33-like Siglecs will slow or stop the growth of human xenotransplants in humanized mouse models.

Antibodies useful in the practice of the invention can be tested in mouse models, e.g., humanized mouse models, using transgenic mice that human CD33-like Siglecs. A transgenic mouse expressing human Siglec-9 under the LysM promoter, which expresses Siglec-9 in myelomonocytic cells in a C57BL/6 background, is described in Laubli et al. (2014) PNAS 111(39):14211. Similar mice can be generated that express other human CD33-like Siglecs, e.g., Siglec-7, in defined cell lineages using known techniques.

Tumor growth or metastasis can be assayed in transgenic mice as follows. Cells derived from C57BL/6 murine colon adenocarcinoma cells (MC-38 cells) are washed in PBS, counted, and resuspended in cold PBS at a concentration of 250,000 viable cells/100 μl. Animals are prepared as needed for injection using standard approved anesthesia. 100 μl of the cell suspension containing 250,000 cells in PBS are subcutaneously injected into the rear flank of the C57 BL/6 transgenic mouse at 6-8 weeks of age.

Animals are monitored every day for palpable tumors, or any changes in appearance or behavior. Once tumors are palpable, tumor size, body weights, and clinical observations are measured 2 times tumors. Tumor size is measured using calipers, and tumor volume will be calculated using the following equation: (longest diameter*shortest diameter²)/2.

When tumor volume reaches 60-80 mm³, mice are randomized into groups of at least 8 animals per group and treated with antibodies directed to a CD-33 Siglec, e.g., anti-Siglec-7 or anti-Siglec-9 antibodies. Appropriate matched isotype control antibodies are used as controls. Antibodies are dosed via intraperitoneal injection 2 times per week for the duration of the study.

The dosing regimen for the test and control antibodies may be 0.1 to 10 mg/kg. Dosing holidays are given at body weight loss of greater than 15%, and dosing resumes when body weight loss is less than 10%. Animals are terminated if tumor size measures greater than 2000 mm³, or if the animal has lost greater than 20% of its pre-treatment body weight.

### INCORPORATION BY REFERENCE

The entire disclosure of each of the patent documents and scientific articles cited herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

1. A method of treating cancer in a subject in need thereof, the method comprising administering to the subject a therapeutically effective amount of an anti-Siglec-7 or anti-Siglec-9 antibody thereby to treat the cancer, wherein the antibody interferes with the binding of Siglec-7 or Siglec-9 to its corresponding sialic acid ligand.

2. The method of claim 1, wherein the antibody interferes with the suppression of cells of the innate immune response directed to the cancer.

3. The method of claim 1, wherein the Siglec-7 or Siglec-9 is expressed on the surface of a cell of the innate immune system.

4. The method of claim 1, wherein the sialic acid ligand is a sialoglycoprotein expressed on the surface of a cancer cell.

5. The method of claim 1, wherein the sialic acid ligand is a sialoglycoprotein secreted by a cancer cell.

6. The method of claim 1, wherein the antibody that binds Siglec-7 binds to an epitope selected from the group consisting of amino acid residues 19-32 of SEQ ID NO: 1, amino acid residues 47-76 of SEQ ID NO: 1, amino acid residues 47-62 of SEQ ID NO: 1 and amino acid residues 63-76 of SEQ ID NO: 1.

7. The method of claim 1, wherein the antibody that binds Siglec-9 binds to an epitope selected from the group consisting of amino acid residues 18-27 of SEQ ID NO: 2, amino acid residues 42-72 of SEQ ID NO: 2, amino acid residues 42-58 of SEQ ID NO: 2, amino acid residues 59-72 of SEQ ID NO: 2, amino acid residues 20-33 of SEQ ID NO: 3, amino acid residues 48-77 of SEQ ID NO: 3, amino acid residues 48-63 of SEQ ID NO: 3, and amino acid residues 64-77 of SEQ ID NO:3.

8. The method of claim 1, wherein the sialic acid ligand is a mucin.

9. The method of claim 2, wherein the cells of the innate immune system are myeloid progenitors, monocytes, neutrophils, NK cells, dendritic cells, macrophages, or eosinophils.

10. The method of claim 1, wherein the cancer is an epithelial cancer or a carcinoma.

11. The method of claim 10, wherein the epithelial cancer is endometrial cancer, ovarian cancer, cervical cancer, vulvar cancer, uterine cancer, fallopian tube cancer, breast cancer, prostate cancer, lung cancer, pancreatic cancer, urinary cancer, bladder cancer, head and neck cancer, oral cancer or liver cancer.

12. The method of claim 10, wherein the carcinoma is an acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, baso squamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, and carcinoma villosum.
